(19) United States
(12) Patent Application Publication
(10) Pub. No.: US 2019/0059860 A1
(43) Pub. Date: Feb. 28, 2019

(54) STOOL SPECIMEN COLLECTING, SAMPLING AND DIAGNOSING MEANS AND METHODS THEREOF

(71) Applicant: 4C Diagnostics Ltd., Haifa (IL)

(72) Inventors: Daniel SHAHAF, Kibbutz Degania (IL); David NAPCHI, Savyon (IL); Eyal BRESSLER, Ramat-Gan (IL); Iris SHICHOR, Zikhron-Yaakov (IL); Sharon BEN_YEHUDA, Rechovot (IL)

(73) Assignee: 4C Diagnostics Ltd., Haifa (IL)

(21) Appl. No.: 15/761,888

(22) PCT Filed: Sep. 21, 2016
(86) PCT No.: PCT/IL2016/051044

§ 371 Date: Mar. 21, 2018

Related U.S. Application Data
(60) Provisional application No. 62/377,620, filed on Aug. 21, 2016.
(60) Provisional application No. 62/221,127, filed on Sep. 21, 2015.

(51) Int. Cl. A61B 10/00; G01N 1/02; G01N 33/68; G01N 21/78

(52) U.S. Cl. CPC A61B 10/0038; G01N 1/02; G01N 2021/7759; G01N 21/78; G01N 2001/028; G01N 33/6893

(57) ABSTRACT

System, means and methods are provided for collecting, sampling and testing stool samples. The present invention pertains to both mechanical and electrical, manual, semi-automatic, automatic means and methods for automatic sampling and homogenization or extraction of the stool samples, enabling their usability in various diagnosis and detection applications.

### FIELD OF THE INVENTION

The present invention pertains to a stool specimen collecting, sampling and diagnosing systems, means, kits, and methods thereof. More specifically, the invention relates to a single use, disposable, intuitively operated stool collecting, sampling and diagnosing modules and kit thereof, and to methods of manufacturing, collecting stool, sampling and diagnosing the same.

### BACKGROUND OF THE INVENTION

Jones et al. states that foodborne diseases cause an estimated 76 million illnesses and 5,000 deaths in the United States each year (2001). In 68% of foodborne disease outbreaks, no etiologic pathogen is identified. In two-thirds of outbreaks with no identified etiology, no stool specimens are submitted for testing. In the absence of adequate stool specimens, improved laboratory testing techniques and enhanced surveillance activities cannot increase the proportion of outbreaks of infection with a confirmed etiology. They also concluded that potential barriers to successful collection of stool specimens include delays in requesting specimen collection from ill persons, lack of easy-to-use collection materials and instructions, and inconvenience associated with storing and transporting specimens to the laboratory, See Jones et al., Use of Stool Collection Kits Delivered to Patients Can Improve Confirmation of Etiology in Foodborne Disease Outbreaks; Clin Infect Dis. (2004) 39 (10): 1454-1459, which is disclosed herein as a reference.

U.S. Pat. No. 6,351,857, which is disclosed herein as a reference, discloses that a significant problem with using stool specimens in screening or diagnosis arises in the collection of the specimen. Depending on its physical state, the specimen may be difficult to collect into a container. Moreover, the handling of a specimen raises sanitary concerns. Merely obtaining a specimen may increase the risk of disease transmission, especially if sanitary procedures are not followed. This is especially true if the patient is responsible for obtaining the specimen. The odor often associated with a stool specimen, and the frequent need to transport the specimen, also contribute to the problems associated with sample collection.

Likewise, US patent application 2003014705 which is disclosed herein as a reference, underlines that as known by the professionals of the area, in order to perform a laboratory diagnosis, when the collection of more than one specimen is required, such as stool parasitological examinations, the user should collect the stool specimen on three alternate days and have them stored in a corresponding sterile pot available from drugstores or furnished by the laboratory itself. Although this system of stool collection and delivery to the laboratory is a reasonably satisfactory practice, it poses some inconveniences, to the extent that it makes collection embarrassing to the user and specimen handling difficult to the laboratory personnel. Another inconvenience found in this usual practice of stool collection for laboratory examination refers to the use of the traditionally adopted pots, which implies a high degree of contamination as a result of inappropriate handling at the time of stool collection, as well as at the time the specimens collected are transferred to the specimen concentrator container, which remains at the lab. Still a third problem identified in this usual practice of stool collection refers to the form of the pots for specimen collection, which require that the specimen is stored with the use of specific tools, both by the user at the time of collection and by the lab technician for the transfer of specimens collected to an already standardized laboratory system to process them. Examinations for the diagnosis of intestinal parasitosis usually involve handling of fecal specimen. More practical kits, however, are available in the market, with the capacity to process only one stool specimen, for instance, Coprotest, a trade name owned by NL Comercio Exterior Ltda. 3, the single kit currently marketed in the country, and others marketed only abroad, such as ParaSep, a trademark registered to DiaSys Co. 4, FPCJumbo, a trademark registered to Evergreen Scientific Co., ParaSed, trademark pertaining to Medical Chemical Co., and Macro-Corn, a trademark owned by Meredian Diagnostic Inc.

US application 20120276059, which is incorporated herein as a reference, discloses a method of monitoring a patient with C. Difficile disease, the method comprising: obtaining a first fecal sample from a patient at a first time; obtaining a second fecal sample from the same patient at a second time later than the first time; comparing a first amount of one or more of lactoferrin or calprotectin in the first fecal sample with a second amount of one or more of lactoferrin or calprotectin in the second fecal sample to identify a change in level of one or more of lactoferrin or calprotectin between the first time and the second time; based on a change in level of one or more of lactoferrin or calprotectin, identifying a patient's change in C. Difficile disease severity; and administering a therapeutically effective amount of a treatment shown to be effective in treating C. Difficile to the patient based on identifying a patient's change in C. Difficile disease severity.

For Crohn's disease (CD) and ulcerative colitis (UC), and other inflammatory bowel diseases (IBD), early steps in the diagnostic process include laboratory tests of blood and fecal matter. Stool specimens are analyzed to eliminate the possibility of bacterial, viral, or parasitic causes of diarrhea. Blood tests can check for signs of infection as well as for anemia, which may indicate bleeding in the colon or rectum. Mylonaki et al., report that during the period 1997-2001 there were 237 relapses in 213 patients. Enteric infection was found in 25 (10.5%) relapses in 24 patients; in seven patients, infection was associated with the initial presentation of their IBD. Clostridium difficile toxin was detected in 13 (5.5%) instances; the 12 other infections (5% relapses) were Campylobacter spp. (five), Entamoeba histolytica (three), Salmonella spp. (one), Plesiomonas shigelloides (one), Strongyloides stercoralis (one) and Blastocystis hominis (one). There was a significant association between infection and the need for hospital admission. Of the 13 relapses associated with C. difficile, ten were in outpatients, seven patients had undergone previous antibiotic treatment, and four patients were presenting with IBD for the first time. All relapses resolved satisfactorily after treatment with antibiotics with or without corticosteroids, see Enteric infection in relapse of inflammatory bowel disease: importance of microbiological examination of stool Mylonaki, et al., European Journal of Gastroenterology & Hepatology, August 2004, 16(8):775-778 which is incoprporated herein as a reference.

Langhorst et al. compare the performance of fecal lactoferrin (Lf), calprotectin (Cal), polymorphonuclear neutrophil elastase (PMN-e), as well as serum C-reactive protein (CRP) in patients with IBD to address (a) whether these markers can differentiate IBD patients with endoscopically assessed inflammation from IBD patients without inflammation and from irritable bowel syndrome (IBS); (b) whether they correlate with endoscopic severity of inflammation; and (c) whether a combination of fecal markers with the respective disease-specific activity indices may increase the diagnostic accuracy with reference to the endoscopic severity of inflammation. They show that fecal markers Lf, Cal, and PMN-e are able to differentiate active BD from inactive IBD as well as from IBS. None of these three stool markers is consistently superior in its ability to reflect endoscopic inflammation, but all three are superior to CRP in their diagnostic accuracy. A combination of the stool markers with the CRP and a disease-specific activity index in a categorical comprehensive activity index increases the diagnostic accuracy with reference to the endoscopic inflammation in UC, See Langhorst et al., Noninvasive Markers in the Assessment of Intestinal Inflammation in Inflammatory Bowel Diseases: Performance of Fecal Lactoferrin, Calprotectin, and PMN-Elastase, CRP, and Clinical Indices. Am J Gastroenterol. (2008) 103(1) 162-9, which is incorporated herein as a reference. US application 20110212104, which is further incorporated herein as a reference, discloses biomarkers associated with inflammatory bowel disease (IBD), as well as methods of using such biomarkers for diagnosing, assessing and monitoring disease progression. The biomarkers are measured at the protein level or the gene expression level, and tracked individually or in groups of two or more. The disclosed biomarkers are useful for monitoring a course of therapy, such as treatment with an IL-23 antagonist. Changes in biomarker levels are used to confirm target engagement and therapeutic efficacy and to inform modification of a therapeutic regimen, for example to increase or decrease dosing of a therapeutic agent, such as an anti-IL-23 or anti-IL-23R antibody.

It is nevertheless still an unmet need for a single use, disposable, automatically and intuitively operated stools collecting, sampling and diagnosing modules and kits thereof, and to methods of manufacturing, collecting stool specimens, sampling and instantaneously diagnosing the same.

### SUMMARY OF THE INVENTION

It is thus one object of the present invention to provide a stool sample collecting and diagnosing device, characterized by a first module comprising a motor actuating a rotatable axle; and, a second module comprising: a rotatable shaft reversibly connectable with said rotatable axle; said shaft is further connected to and wounded by an elongated cord by the proximal portion thereof; the distal portion of said cord comprises means for retaining stool material; a pierceable chamber with predefined volume of stool-sample homogenizing fluid; a stool-sampling cabinet of a predefined volume throughout which said cord is crossing from, configured to accommodate a meter dosed stool sample; and, a diagnosing strip to which a predefined small volume of stool sample is introduced after being wet by said homogenizing fluid.

It is another object of the present invention to provide an apparatus for collecting, homogenizing and/or extracting a stool specimen, comprising: a housing comprising a homogenization chamber containing an extraction-homogenization buffer; a retractable swab having proximal and distal opposite ends; characterized by at least 2 configurations; an EXTENDED configuration, in which said distal end is configured to be in contact with said stool specimen; and, a RETRACTED configuration, in which said swab's distal end is retracted into said homogenization chamber configuration; wherein said retractable swab is converted into said RETRACTED configuration to retract said swab's distal end into said homogenization chamber while said extraction-homogenization buffer is whirled said with said stool specimen; so as to result in homogenized and/or extracted stool.

It is another object of the present invention to provide an apparatus as defined in any of the above, the apparatus additionally comprising a dripping mechanism configured to provide application of said homogenized and/or extracted stool onto an external surface.

It is another object of the present invention to provide an apparatus as defined in any of the above, the apparatus said external surface is a diagnostic strip.

It is another object of the present invention to provide an apparatus as defined in any of the above, wherein said retractable swab can be transformed from said EXTENDED configuration to said RETRACTED configuration and/or vice versa.

It is another object of the present invention to provide an apparatus as defined in any of the above, wherein in said RETRACTED configuration, said swab is spirally-wounded.

It is another object of the present invention to provide an apparatus as defined in any of the above, wherein said homogenization chamber comprises a predefined volume of extraction-homogenization buffer, for diluting said sampled stool specimen.

It is another object of the present invention to provide an apparatus as defined in any of the above, wherein said retractable swab is made of or otherwise comprising at least one material selected from a group consisting of an abrasive material, an absorbing material, porosive material, layered and multi-laminar material, emulsified material, liposomal material, salt-containing material, buffer-containing material, aggregated material, solid particles, fibers-containing material, filament(s)-containing material, pre-moistened material and any combination thereof.

It is another object of the present invention to provide an apparatus as defined in any of the above, wherein said retractable swab is in the shape of a string, a threaded rope, natural fiber rope, synthetic fiber rope, tube-like, sponge-like thread, projected bulges, a vertebrate-like structure, cord-like thick and/or hairy fiber, dental-floss-like filament having a thin and narrow cross section; filament having grooves, measuring tape-like swab having relatively wide and thin cross section and any combination thereof, an

It is another object of the present invention to provide an apparatus as defined in any of the above, further it comprising one or more stool resecting lips (SRLs); said SRLs are configured to be in contact with at least one surface of said retractable swab, and provided to resect surplus stool attached to said retractable swab when deployed from its EXTENDED configuration to its RETRACTED configuration, thereby providing meter-dose sample within said housing.

It is another object of the present invention to provide an apparatus as defined in any of the above, wherein said SRLs are affixed in said housing, provided outside said housing, provided along said retractable swab and any combination thereof.

It is another object of the present invention to provide an apparatus as defined in any of the above, wherein said SLRs are in a shape selected from a group consisting of a one directional valve, a slit-like opening, a round-like opening, a duck-beak-like shape and any combination thereof.

It is another object of the present invention to provide an apparatus as defined in any of the above, wherein said SLRs are configured to be modified to resect said meter-dosed sample in a plurality of sample sizes.

It is another object of the present invention to provide an apparatus as defined in any of the above, it comprises at least one detector for testing said stool specimen.

It is another object of the present invention to provide an apparatus as defined in any of the above, wherein said detector is directed towards a marker selected from the group consisting of Calprotectin, Lactofferin, S100A12 and any combination thereof.

It is another object of the present invention to provide an apparatus as defined in any of the above, wherein said detector is directed towards a marker selected from the group consisting of Pancreatic Elastase Anti-Gliadin-IgA-Antibodies, M2PK, sDNA, C. difficile bacteria, Helicobacter pylori, parasites, Candida, leucocytes, Rota virus, Chlamydia, Salmonella, PH and any combination thereof.

It is another object of the present invention to provide an apparatus as defined in any of the above, wherein said detector is a lateral flow strip configured in a shape selected from the group consisting of a linear strip, a rectangle, a circle, a triangle, a polygon and any combination thereof.

It is another object of the present invention to provide an apparatus as defined in any of the above, it comprising at least two detectors provided in variable diameters, and configured to be organized within one another in the same plane.

It is another object of the present invention to provide an apparatus as defined in any of the above, wherein said detector is barriered from said homogenization chamber by means of a sealing cover having a release member characterized by an OFF configuration barring fluid communication between said homogenization chamber and said detector, and an ON configuration allowing fluid communication between said homogenization chamber and said detector.

It is another object of the present invention to provide an apparatus as defined in any of the above, wherein said detector is barriered from said homogenization chamber by means of a separating membrane which is configured to selectively facilitate buffer's influx while it effectively blocks stool contact with said detector.

It is another object of the present invention to provide an apparatus as defined in any of the above, the apparatus further comprising a diagnosis cover, configured to at least partially cover said detector.

It is another object of the present invention to provide an apparatus as defined in any of the above, wherein at least part of said diagnosis cover is transparent, enabling viewing of said detector.

It is another object of the present invention to provide an apparatus as defined in any of the above, the apparatus further comprising a swab-conducting tube interconnected to said housing providing a retraction passage of said retractable swab when deployed from its EXTENDED configuration to its RETRACTED configuration, and/or vice versa.

It is another object of the present invention to provide an apparatus as defined in any of the above, wherein said retractable swab's distal end further comprises a cap configured to seal said retractable swab's entry point in said housing.

It is another object of the present invention to provide an apparatus as defined in any of the above, wherein it comprising an operation button operatively connected to said spring mechanism.

It is another object of the present invention to provide an apparatus as defined in any of the above, wherein said operation button is in the form of a pin, a safety catch, clasp, knob, clamp, snap, latch, bolt and any combination thereof.

It is another object of the present invention to provide an apparatus as defined in any of the above, wherein said operation button is operated by pressing, pushing, pulling, twisting, sliding and any combination thereof.

It is another object of the present invention to provide an apparatus as defined in any of the above, wherein the apparatus further comprising at least one elastomeric gasket provided for sealing.

It is another object of the present invention to provide an apparatus as defined in any of the above, wherein said extraction-homogenization buffer is provided in a sack-like compartment located inside said homogenization chamber.

It is another object of the present invention to provide an apparatus as defined in any of the above, wherein it further comprising a stool collector configured to be reversibly installed within a toilet-bowl and comprising a stool specimen collecting zone, said stool collector is connected to said housing and configured to accommodate said retractable swab's distal end in said collection zone, thereby facilitating said contact with said stool specimen.

It is another object of the present invention to provide an apparatus as defined in any of the above, wherein said stool collector is characterized by a nest-shaped structure comprising an upper opening, at least partially encircled by a toilet-attaching circumference, and a concave central portion, interconnected to said circumference, said central portion configured for confining said stool specimen.

It is another object of the present invention to provide an apparatus as defined in any of the above, the apparatus is characterized by an elongated structure and comprising: a first open bore body, having an applicable volume (Vn), at distal end, configured for extracting said retractable string-like swab, thereby extracting a metered dose (Vs) stool's specimen; and a second open-bored body, in a fluid connection with said first open bore body via an aperture, comprising: a predefined volume confined in said envelope, containing a predefined volume of a buffer, for diluting said sampled stool; and at least one detector for testing said buffer-admixed stool specimen.

It is another object of the present invention to provide an apparatus as defined in any of the above, wherein it further comprising a pumping mechanism located at said second open-bored body, configured to provide said body with an effective vacuum to influx said metered dose (Vs) stool's specimen, temporarily accommodated within the first open bore body, and further admix it with the buffer contained in said second open bore body, such that homogenized buffer-diluted stool suspension is obtained.

It is another object of the present invention to provide a method for collecting, homogenizing and/or extracting a stool specimen, characterized by steps of providing a first module comprising a motor actuating a rotatable axle; providing a second module comprising: a rotatable shaft reversibly connectable with said rotatable axle; said shaft is further connected to and wounded by an elongated cord by the proximal portion thereof; the distal portion of said cord comprises means for retaining stool material; providing a pierceable chamber with predefined volume of stool-sample homogenizing fluid; providing a stool-sampling cabinet of a predefined volume throughout which said cord is crossing from, configured to accommodate a meter dosed stool sample; and providing a diagnosing strip to which a predefined small volume of stool sample is introduced after being wet by said homogenizing fluid; collecting said meter dosed stool sample within said stool-sampling cabinet; piercing said pierceable chamber; and, mixing said a predefined small volume of stool sample with said homogenizing fluid; thereby collecting, homogenizing and/or extracting said stool specimen.

It is another object of the present invention to provide a method for collecting, homogenizing and/or extracting a stool specimen, characterized by steps of providing an apparatus, comprising: a housing comprising a homogenization chamber containing an extraction-homogenization buffer; a retractable swab having proximal and distal opposite ends; characterized by at least 2 configurations; an EXTENDED configuration, in which said distal end is configured to be in contact with said stool specimen; and, a RETRACTED configuration, in which said swab's distal end is retracted into said homogenization chamber configuration; retracting said retractable swab's distal end into said homogenization chamber, defining a RETRACTED configuration while whirling said extraction-homogenization buffer with said stool specimen, thereby resulting in homogenized and/or extracted stool.

It is another object of the present invention to provide the method as defined in any of the above, wherein the method further comprising steps of connecting said swab's distal end with said spring mechanism and deploying said distal portion from said an EXTENDED configuration to a RETRACTED configuration and/or vice versa.

It is another object of the present invention to provide the method as defined in any of the above, wherein the method further comprising steps of spirally-wounding said swab in said RETRACTED configuration.

It is another object of the present invention to provide the method as defined in any of the above, wherein the method further comprising steps of diluting said sampled stool specimen in a predefined volume of extraction-homogenization buffer.

It is another object of the present invention to provide the method as defined in any of the above, wherein the method, further comprising steps of configuring at least a portion of said swab's distal portion to a shape, and selecting the same from a group consisting of a string, a threaded rope, natural fiber rope, synthetic fiber rope, tube-like, sponge-like thread, projected bulges, a vertebrate-like structure, cord-like thick and/or hairy fiber, dental-floss-like filament having a thin and narrow cross section; measuring tape-like swab having relatively wide and thin cross section and any combination thereof, and further comprising configuring said swab to collect stool in a fluid, solid or semi-solid state.

It is another object of the present invention to provide the method as defined in any of the above, wherein the method further comprising steps of making at least a portion of said swab from at least one material selected from a group consisting of an abrasive material, an absorbing material, porosive material, layered and multi-laminar material, emulsified material, liposomal material, salt-containing material, buffer-containing material, aggregated material, solid particles, fibers-containing material, filament(s)-containing material, pre-moistened material and any combination thereof.

It is another object of the present invention to provide the method as defined in any of the above, wherein the method further comprising steps of incorporating at least one detector in said swab extender.

It is another object of the present invention to provide the method as defined in any of the above, wherein the method further comprising steps of providing at least a portion of said housing to be transparent such that said stool's test, detected by said detector, is visible outside said housing.

It is another object of the present invention to provide the method as defined in any of the above, wherein the method further comprising steps of isolating said homogenization chamber by means of a sealing cover, and providing a release member characterized by an OFF configuration barring fluid communication between said homogenization chamber and said detector, and an ON configuration allowing fluid communication between said homogenization chamber and said detector.

It is another object of the present invention to provide the method as defined in any of the above, wherein the method further comprising steps of separating said buffer-diluted stool by means of a membrane, thereby selectively facilitating buffer's influx while effectively blocking stool contact with said detector.

It is another object of the present invention to provide the method as defined in any of the above, wherein the method further comprising steps of providing said extender (_{housing}) with one or more stool resecting lips (SRLs); affixing said SRLs in said housing to touch at least one surface of said swab, and resecting surplus stool attached to said swab when deploying the same from its EXTENDED configuration to its RETRACTED (_{collapsed}) configuration, thereby providing meter-dose sample within said housing.

It is another object of the present invention to provide the method as defined in any of the above, wherein said step of whirling is provided by a pumping mechanism, a flange-stirring mechanism, a blender-like stirring mechanism and any combination thereof.

It is another object of the present invention to provide an apparatus for collecting, homogenizing and/or extracting a stool specimen, comprising: a housing comprising a homogenization chamber containing an extraction-homogenization buffer; a retraction mechanism positioned in contact with said homogenization chamber; and a retractable swab having proximal and distal opposite ends, said proximal end is configured to be in contact with said retraction mechanism and said distal end is configured to be in contact with said stool specimen, thereby defining an EXTENDED configuration; wherein said retraction mechanism is configured to retract said retractable swab's distal end into said homogenization chamber, thereby defining a RETRACTED configuration, while providing whirling of said extraction-homogenization buffer with said stool specimen, thereby resulting in homogenized stool.

It is another object of the present invention to provide the apparatus as defined in any of the above, wherein retraction mechanism comprises one or more members of a group consisting of a spring, a vacuum pump, a pneumatic motor, an electric motor, a manual motor and any combination thereof.

It is another object of the present invention to provide a system for collecting stool specimen, comprising: a stool collector configured to be reversibly installed within a toilet-bowl, characterized by a nest-shaped structure comprising an upper opening, at least partially encircled by a toilet-attaching circumference, and a concave central portion, interconnected to said circumference, said central portion configured for confining stool's specimen; a retractable string-like swab having a distal and proximal opposite ends, said distal end positioned in said concave central portion and configured to be in contact with said stool, and said proximal end positioned outside of said concave central portion; and a swab extender (housing) configured to be in connection with said stool collector; wherein said swab's proximal end is positioned in said swab extender and said distal end is deployable in two alternative configurations, namely EXTENDED configuration and RETRACTED (_{collapsed}) configuration.

It is another object of the present invention to provide a method for collecting stool specimen, comprising steps of: providing a system comprising: a stool collector configured to be reversibly installed within a toilet-bowl, characterized by a nest-shaped structure comprising an upper opening, at least partially encircled by a toilet-attaching circumference, and a concave central portion, interconnected to said circumference, said central portion configured for confining stool's specimen; a retractable string-like swab having a distal and proximal opposite ends, said distal end positioned in said concave central portion and configured to be in contact with said stool, and said proximal end positioned outside of said concave central portion; and a swab extender (housing) configured to be in connection with said stool collector; wherein said swab's proximal end is positioned in said swab extender and said distal end is deployable in two alternative configurations, namely EXTENDED configuration and RETRACTED (collapsed) configuration; positioning and securing said stool collector onto a toilet facility; evacuating a stool into said concave central portion containing said retractable string-like swab; thereby collecting stool specimen; and retracting said retractable string-like swab into said swab extender (housing).

It is another object of the present invention to provide a stool collector configured to be reversibly installed within a toilet-bowl, characterized by a nest-shaped structure comprising an upper opening, at least partially encircled by a toilet-attaching circumference, and a concave central portion, interconnected to said circumference, said central portion configured for confining stool's specimen.

It is another object of the present invention to provide a stool collector as defined in any of the above, wherein said toilet-attaching circumference comprises one or more (a) elastic portions and/or (b) flaps, configured to fasten said collector, by means of (a) said elastic portions and/or (b) flaps, to the toilet's bowl, toilet seat or a combination thereof.

It is another object of the present invention to provide a stool collector as defined in any of the above, wherein said toilet-attaching circumference, along one or more portions thereof, comprises or otherwise provided in connection with a rope, configured to fasten said collector, by means of said rope, to the toilet's bowl, toilet seat or a combination thereof.

It is another object of the present invention to provide a stool collector as defined in any of the above, wherein said toilet-attaching circumference, along one or more portions thereof, comprises or otherwise provided in connection with a water-miscible glue, configured to fasten said collector, by means of said glue, to the toilet's bowl, toilet seat or a combination thereof.

It is another object of the present invention to provide a stool collector as defined in any of the above, wherein said collector further comprises a continuous envelope configured to connect said central portion to said toilet-attaching circumference in a continuous manner.

It is another object of the present invention to provide a stool collector as defined in any of the above, wherein at least a portion of said collector entirely obstructing the passage of light.

It is another object of the present invention to provide a stool collector as defined in any of the above, wherein said collector further comprises a net-like perforated envelope configured to connect said central portion to said toilet-attaching circumference in a non-continuous manner.

It is another object of the present invention to provide a stool collector as defined in any of the above, wherein said collector further comprises in, on or in connection with said central portion stool specimen immobilizing means (SSIM).

It is another object of the present invention to provide a stool collector as defined in any of the above, wherein said SSIM is selected form a group consisting of an abrasive material, an absorbing material, porosive material, layered and multi-laminar material, emulsion, liposomal, salt-containing material, buffer-containing material, aggregates and solid particales, fibers-containing matter, filament(s)-containing material, premoistened material, spiral-wound dressing and a combination thereof.

It is another object of the present invention to provide a stool collector as defined in any of the above, wherein it made of one or more members of a group consisting of pre-moistened material, flushable towelette, flushable towelette impregnated with polyvinyl alcohol-containing binders, non-woven web and a patterned sheet thereof, toilet paper and the like, toilet tissues, tissues, wipes in various grades, a paper-made patterned sheet, polymeric material, especially polylactic and polyglutamic acid-containing polymers, polyamide-containing polymers and any combination thereof.

It is another object of the present invention to provide a stool collector as defined in any of the above, wherein said collector further comprises a pierceable surface for collecting a stool specimen form said central portion.

It is another object of the present invention to provide a stool collector as defined in any of the above, wherein it characterized by an OPEN configuration where the collector is attached to the toilet with its upper opening being accessible, and a CLOSE configuration where the collector, by means of said circumference or attachment thereof, is tightly sealed such that a stool is securely contained within, thereby providing a closeable stool collector.

It is another object of the present invention to provide a sampler, characterized by an elongated syringe-shaped structure characterized by an open bore needle, having an applicable volume (Vn), at distal end, for piercing a pierceable surface of a stool-containing collector, thereby extracting a metered dose (Vs) stool's specimen, Vn equals Vs; and an elongated open-bored body, in a fluid connection with said needle via a needle-inlet provided in said body's envelope, comprising a predefined volume confined in said envelope, said body contains a predefined volume of a buffer, for diluting said sampled stool; and at least one detector for testing said buffer-admixed stool specimen.

It is another object of the present invention to provide a stool sampler as defined in any of the above, wherein at least a portion of said envelope is transparent such that said stool's test, detected by said detector, is visible outside said sampler.

It is another object of the present invention to provide a stool sampler as defined in any of the above, wherein each of which of said at least one detector is correlated with a detection indication window encompassing at least a portion of said envelope.

It is another object of the present invention to provide a stool sampler as defined in any of the above, wherein each of which of said indication windows illustrates positive or negative detection in the form selected from the group consisting of a change of color, a change of color intensity, appearance of a marker, and any combination thereof.

It is another object of the present invention to provide a stool sampler as defined in any of the above, wherein said detector is barriered from said buffer-diluted stool by means of a separating membrane which is configured to selectively facilitate buffer's influx while it effectively blocks stool contact with said detector.

It is another object of the present invention to provide a stool sampler as defined in any of the above, wherein said needle is reciprocally moveable along sampler's main longitudinal axis, wherein said needle, and sampled stool within, is configured to sealably enter said sampler body via said needle-inlet whilst effectively blocking buffer's efflux.

It is another object of the present invention to provide a stool sampler as defined in any of the above, wherein said sampler further comprising a pumping mechanism located at said sampler's proximal side, thereby resulting in a homogenizing stool sampler.

It is another object of the present invention to provide a stool sampler as above, wherein at least one of the following is being held true: said pumping mechanism is configured to provide said body with an effective vacuum to influx said metered dose (Vs) stool's specimen, temporarily accommodated within the pierced needle, and further admix it with the buffer, such that homogenized buffer-diluted stool suspension is obtained; a pumping mechanism is configured to provide said body with an effective vacuum to reciprocally suck said needle, and sampled stool within, along sampler's main longitudinal axis into said body; a pumping mechanism is configured to first (a) provide said body with an effective vacuum to withdraw said stool specimen out from said needle bore, and then (b) admix said stool with said buffer until a homogenized suspension is obtained; or said pumping mechanism is configured to provide said body with an effective vacuum to facilitate the efflux of said buffer via said membrane towards said detector.

It is another object of the present invention to provide a method of stool sampling, characterized by step of providing an elongated syringe-shaped structure with an open bore needle, having an applicable volume (Vn), at distal end, for piercing a pierceable surface of a stool-containing collector, thereby extracting a metered dose (Vs) stool's specimen, Vn equals Vs; and further providing an elongated open-bored body in a fluid connection with said needle via a needle-inlet provided in said body's envelope, comprising a predefined volume confined in said envelope; containing, in said body, a predefined volume of a buffer, thereby diluting said sampled stool; and further providing the same with at least one detector thereby testing said buffer-admixed stool specimen.

It is another object of the present invention to provide a kit for collecting, sampling and testing stool specimen, comprising: a stool collector configured to be reversibly installed within a toilet-bowl, characterized by a nest-shaped structure comprising an upper opening, at least partially encircled by a toilet-attaching circumference, and a concave central portion, interconnected to said circumference, said central portion configured for confining stool's specimen; and one member of a group consisting of: a stool sampler, characterized by an elongated syringe-shaped structure characterized by an open bore needle, having an applicable volume (Vn), at distal end, for piercing a pierceable surface of a stool-containing collector, thereby extracting a metered dose (Vs) stool's specimen, Vn equals Vs; and an elongated open-bored body, in a fluid connection with said needle via a needle-inlet provided in said body's envelope, comprising a predefined volume confined in said envelope, said body contains a predefined volume of a buffer, for diluting said sampled stool; and at least one detector for testing said buffer-admixed stool specimen; a retractable stool's specimen swab, characterized by an endless thin stool's swab having a distal and proximal opposite ends, said proximal end is attached to a swab′ extender (housing); said distal end is deployable in two alternative configurations, namely EXTENDED configuration and RETRACTED (collapsed) configuration; and a stool testing retractable swab, characterized by said endless thin stool's swab, wherein said swab′ extender (housing) further comprising a detector for testing said stool specimen; configurations, namely an EXTENDED configuration and a RETRACTED (collapsed) configuration; and a stool testing retractable swab, with an endless thin stool's swab member, providing said swab′ extender (housing) with a detector, and testing said stool specimen by mean of said detector.

It is another object of the present invention to provide a system for collecting, homogenizing and/or extracting, and diagnosing a stool specimen, comprising: an apparatus comprising: a housing comprising a homogenization chamber containing an extraction-homogenization buffer; a spring mechanism or an equivalent motor; a retractable swab having proximal and distal opposite ends, said proximal end is configured to be in contact with said spring mechanism and said distal end is configured to be in contact with said stool specimen, defining an EXTENDED configuration; and at least one detector; and an imaging means configured to image said detector; wherein said spring mechanism is configured to retract said retractable swab's distal end into said homogenization chamber, defining a RETRACTED configuration, while providing whirling of said extraction-homogenization buffer with said stool specimen, thereby resulting in homogenized and/or extracted stool; further wherein said at least one detector is configured for testing said homogenized and/or extracted stool.

It is another object of the present invention to provide the system as defined above, wherein it further comprising a processor configured to read a computer readable medium with instructions for: obtaining said image of said detector from said imaging means; calculating according to said image a quantitative estimate of a parameter of said detector; and optionally communicate said image or said estimate to an electronic device.

It is another object of the present invention to provide the system as defined in any of the above, wherein said parameter is selected from the group consisting of result detection, result intensity, control detection and any combination thereof.\

It is another object of the present invention to provide the system as defined in any of the above, wherein said electronic device is selected from the group consisting of a server, a mobile phone, a tablet, a personal computer, a cloud-like server and any combination thereof.

It is another object of the present invention to provide a system for collecting, homogenizing and/or extracting, and diagnosing a stool specimen, comprising: an apparatus comprising: a housing having a window; a spring mechanism or an equivalent motor arrangment; and a retractable swab having proximal and distal opposite ends, said proximal end is configured to be in contact with said spring mechanism and said distal end is configured to be in contact with said stool specimen, defining an EXTENDED configuration; an imaging means; wherein said spring mechanism is configured to retract said retractable swab's distal end into said housing, defining a RETRACTED configuration; further wherein said at least one window is configured for viewing said stool specimen, and said imaging means is configured for imaging said stool specimen through said window.

It is another object of the present invention to provide the system as defined in any of the above, wherein it further comprising a processor configured to read a computer readable medium with instructions for: obtaining said image of said stool specimen; calculating according to said image a quantitative estimate of a parameter of said stool specimen; and optionally communicating said image or said estimate to an electronic device.

It is another object of the present invention to provide the system as defined in any of the above, wherein said parameter is selected from the group consisting of stool specimen color, stool specimen consistency and any combination thereof.

It is another object of the present invention to provide the system as defined in any of the above, wherein said electronic device is selected from the group consisting of a server, a mobile phone, a tablet, a personal computer, a cloud-like server and any combination thereof.

It is another object of the present invention to provide the system as defined in any of the above, additionally comprising a spring mechanism in communication with said proximal end of said retractable swab.

### BRIEF DESCRIPTION OF THE FIGURES

The foregoing features, as well as further objects, advantages, features and characteristics of the present invention, in addition to methods of operation, function of related elements of structure, and the combination of parts and economies of manufacture, generally illustrates system, apparatus, means and methods which are provided useful for collecting, sampling and testing stool samples. This technology pertains to both mechanical and electrical, manual, semi-automatic, automatic means and methods for automatic sampling and homogenization or extraction of the stool samples, enabling their usability in various diagnosis and detection applications.

will become apparent upon consideration of the following description and claims with reference to the accompanying drawing, which forms a part of this specification, wherein:

FIG. 1 schematically illustrates a perspective three-dimensional view of a first embodiment of the present invention, providing a retractable swab, along with a retraction or spring-like mechanism and a housing;

FIGS. 2a and b schematically illustrate a top view and a side view cross-section, respectively, of an embodiment of the present invention, comprising the apparatus disclosed further comprising a detector;

FIGS. 3a and b schematically illustrate a side view and a cross-section of the side view, respectively, presenting the apparatus as shown in FIG. 2;

FIG. 4 schematically illustrates a bottom view of the embodiment of the apparatus as disclosed by the present invention;

FIG. 5 schematically illustrates a three-dimensional explosive view of an embodiment of the apparatus of the present invention;

FIG. 6 schematically illustrates a three-dimensional perspective view of the retractable swab having various embodiments, as disclosed by the present invention;

FIG. 7a-d illustrate a top view, a side cross-section, an explosive three-dimensional view and a close-up view of the retraction or spring-like mechanism as provided in the present invention;

FIG. 8a-f illustrate a bottom view, a side cross-section, a close-up cross-section, a perspective view and an explosive view of an embodiment of the present invention having a release mechanism for allowing fluid communication between the chambers of the housing and the detector;

FIGS. 9a and b schematically illustrate a perspective and a close-up views, respectively, of an embodiment of the present invention provided in the apparatus being connected to a stool collector;

FIGS. 10a and b illustrate two cross-section views of a three-dimensional perspective view of an embodiment of the apparatus as disclosed herein;

FIG. 11 illustrates an out-of-scale side view of the system 100 as disclosed by the present invention;

FIG. 12 illustrates an out-of-scale top view of the system 100 as disclosed by the present invention;

FIG. 13 illustrates a cross-section of the system 100 as disclosed by an embodiment of the present invention;

FIG. 14A-D illustrate in an out-of-scale manner a perspective view of various embodiments of the stool collector as disclosed by the present invention;

FIG. 15A-D illustrate a top view of various embodiments of the stool collector as disclosed by the present invention;

FIG. 16 illustrates a use of the stool collector as disclosed by the present invention;

FIG. 17 illustrates a partial cross-section of a suggested embodiment for using the stool collector as disclosed by the present invention;

FIG. 18A-C schematically illustrate suggested embodiments of the retractable swab positioning in the stool collector.

FIG. 19A-C schematically illustrate suggested embodiments of the extractable swab shape and form, as disclosed by the present invention;

FIG. 20A-B illustrate out-of-scale side view of two suggested embodiments of the swab extender (housing);

FIG. 21A-B illustrate perspective out-of-scale view of two suggested embodiments of the swab extender;

FIG. 22 schematically illustrates an exemplified use of the extractable swab and swab extender having two detectors; and

FIGS. 23-29a and 29b schematically illustrates schematic views of an example of an extractable swab-containing diagnostic tool and kit thereof, comprising a first (e.g., multiuse) module A, and/or a collecting and diagnosing kit comprises both multiuse module A and second (e.g., replaceable or disposable) module B according to an embodiment of the present invention.

### DETAILED DESCRIPTION OF THE PREFERRED EMBODIMENT

The foregoing embodiments discloses as an example system, means and methods are provided for collecting, sampling and testing stool samples, all pertains to both mechanical and electrical, manual, semi-automatic, automatic means and methods for automatic sampling and homogenization or extraction of the stool samples, enabling their usability in various diagnosis and detection applications.

A toilet is a fixture used primarily for the disposal of human urine and feces (stool). In one embodiment of the invention, the term ‘toilet’ refers in a non-limiting manner to the commercially available western vortex-flushing toilet bowl, which creates a self-cleansing effect. The term ‘toilet seat’ refers in a non-limiting manner to a hinged unit consisting of seat, and usually a lid, which is bolted onto a toilet bowl for a flush toilet. A toilet seat may consist of the seat itself, which may be contoured for the user to sit on, and the lid, which covers the toilet when it is not in use—the lid may be absent in some cases, particularly public restrooms.

A detector for analyzing stool specimen, according to the present invention, refers to any means for stool test involves (e.g., biological-, microbial- or chemical-) analysis of fecal matter to diagnose the presence or absence of a medical condition, more specifically, yet in a non-limiting manner, stool test as referred hereinafter, includes biomarkers, microbial marker, chemical markers in general. More specifically, and in a way of example, stool test includes fecal occult blood test, the fecal occult blood test is commonly used to diagnose many conditions that cause bleeding in the gastrointestinal system including colorectal cancer or stomach cancer. Stool tests also include microbiology tests, namely parasitic diseases such as ascariasis, hookworm, strongyloidiasis and whipworm can be diagnosed by examining stools under a microscope for the presence of worm larvae or eggs. Some bacterial diseases can be detected with a stool culture. Toxins from bacteria such as Clostridium difficile (‘C. diff’) can also be identified. Viruses such as rotavirus can also be found in stools. Stool tests further include chemical tests, namely a fecal pH test may be used to determine lactose intolerance or the presence of an infection. Steatorrhea can be diagnosed using a fecal fat test that checks for the malabsorption of fat. Stool tests further include fecal immunochemical test (FIT) and stool DNA test (sDNA), e.g., the PreGen-Plus test, stool guaiac test for fecal occult blood (gFOBT), fecal porphyrin quantification: e.g., HemoQuant. Hence for example, fecal occult blood (FOB) refers to blood in the feces that is not visibly apparent (unlike other types of blood in stool such as melena or hematochezia). A fecal occult blood test (FOBT) checks for hidden (occult) blood in the stool (feces). Similarly, tests may look for globin, DNA, or other blood factors including transferrin, while conventional stool guaiac tests look for heme.

It is an essence of the present invention to provide means and methods for easy and hygienic collection of stool, provided in a retraction/spring-like mechanism which is designed to collect a stool sample and transfer a specimen of it into an extraction-homogenization buffer, with minimal handling and minimal manual operation. The retraction/spring-like mechanism may be provided in some embodiments with a stool collector for directly eliminating stool upon it, and it may also be provided in some embodiments with a built-in detection unit. Thus, in various embodiments, the present invention provides an easily operable and elegant solution for diagnosing stool, by providing a system which enables the steps of the entire diagnostic process—from collecting the stool, through homogenizing it with a buffer and on to the detection processing, without having to manually perform any step.

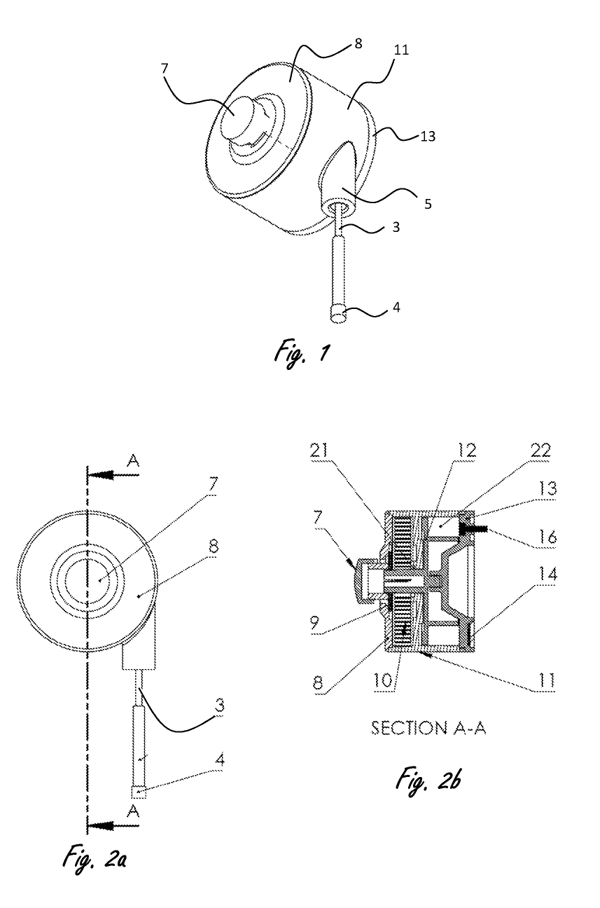

Reference is now made to FIG. 1 schematically presenting a perspective three-dimensional view of a first embodiment of the present invention, providing retractable swab 3, along with retraction/spring-like mechanism 10 provided in housing 11. The retractable swab 3 is configured to have a proximal end connected to retraction/spring-like mechanism 10, found within housing 11, and a distal end which is switchable between two configurations: an extended configuration, having the distal end located outside of housing 11, and a retracted configuration, having the distal end located inside housing 11. In the embodiment shown in FIG. 1, housing 11 is characterized by a round bottle-like shape, having top cover 8 and bottom sealing cover 13. In some embodiments, interconnected to housing 11 is swab-conducting tube 5, which is a tube-like member directing to an aperture in housing 11, and facilitating the transfer of retractable swab 3 from an extended configuration to a retracted configuration and vice versa. Retractable swab 3 may also be provided with cap 4, for sealing and closing the aperture in housing 11, or the aperture in swab-conducting tube 5.

In its extended configuration, retractable swab 3 is designed to be in contact with a stool specimen, and configured to retain at least a portion of the stool on its surface area. In its retracted configuration, retractable swab 3 is designed to be incorporated into housing 11, and specifically to be in contact with homogenization chamber 22, which preferably comprises a homogenizing buffer. The buffer may be provided in some embodiments in a sack-like compartment inside chamber 22. The switch between the extended configuration and the retracted configuration is performed by operating operation button 7, which may be in a non-limiting manner a pin, a safety catch, clasp, knob, clamp, snap, latch, or a bolt and thus may be operated by pressing, pushing, pulling, twisting, sliding and any combination thereof.

It is in accordance with an embodiment of the present invention, wherein the apparatus is configured to provide homogenized stool to an external diagnostic unit. A non-limiting example would be a dripping mechanism which would enable dripping the homogenized stool directly onto a strip provided externally to the apparatus.

Reference is now made to FIGS. 2a and b schematically illustrating a top view and a side view cross-section, respectively, of an embodiment of the present invention, providing the apparatus disclosed further comprising detector 14. The top view illustrated in FIG. 2a exemplifies operation button 7 and top cover 8, and shows retractable swab 3 in at least partially extended configuration, provided outside of swab-conducting tube 5, and provided with cap 4. Line A-A illustrate the cross-section location shown in FIG. 2b. This cross-section view provides the inner configuration of housing 11, which mainly comprises retraction/spring-like mechanism 10, provided in this embodiment as a spring, but which may be in other embodiments, and in a non-limiting example, in the form of a vacuum pump, a pneumatic motor, an electric motor, a manual motor and any combination thereof. Retraction/spring-like mechanism 10 is found in operative connection with retraction release member 9, which is found in operative connection with operation button 7. That is, switching on operation button 7, or in this embodiment pushing it, causes release member 9 to dislocate and to release the gripping of retraction/spring-like mechanism 10, in this embodiment, a spring. The release of spring 10, having the retractable swab 3 connected to it in its proximal end, causes the spiral winding of retractable swab 3, and thus facilitates its switching between the extended configuration and the retracted configuration. The operation of operating button 7 may also lead to fluid communication between the retraction/spring-like mechanism 10 location and the homogenization chamber 22. Thus, extraction-homogenization buffer may reach the retracted swab 3 containing the stool sample. The spiral winding to the stool containing swab 3 inside housing 11 and in contact with the extraction-homogenization buffer leads to the stool's homogenization and completes its preparation for sampling. Bottom cover 12 may isolate retraction/spring-like mechanism 10 from the homogenization chamber 22 having the extraction-homogenization buffer. In some embodiments, bottom cover 12 may be provided with apertures which are configured to be opened only when desiring the operation of the apparatus, and when expecting stool to reach the retraction/spring-like mechanism area.

In an important embodiment of the present invention, the apparatus may be provided with a detection unit comprising of detector 14, preferably provided as a detection strip, and in addition may be also provided with diagnosis cover 15, at least partially covering and protecting detector 14. In various embodiments, at least part of diagnosis cover 15 may also be provided as transparent.

Sealing cover 13 provides isolation to the inner compartments of housing 11, including the homogenization chamber 22 having the extraction-homogenization buffer. In some embodiments, at least one homogenization release member 16 may be incorporated into sealing cover 13, and is configured to be switchable between two configurations: an OFF configuration wherein no fluid communication is provided between homogenization chamber 22 and detector 14, and an ON configuration wherein such fluid communication is allowed. In a preferred embodiment, the shape of homogenization release member 16 is designed such that in the OFF configuration it will mechanically bar and seal aperture 23 provided in sealing cover 13. By dislocating homogenization release member 16 and moving it, the sealing interference is removed and fluid is allowed to pass from homogenization chamber 22, through aperture 23 and into the detection area of detector 14.

In various embodiments, detector 14 may comprise more than one diagnostic strip. Although diagnostic strips may come in this invention in a variety of shapes and polygons, it is preferred to provide detector 14 with a circularly shaped diagnostic strip. Having more than one diagnostic strip, may be in the form of a plurality of torus-shaped strips provided in variable diameters. Having variable diameters enables arranging the diagnostic strips in one plane, having the smallest diameter provided in the center and increasing diameters surrounding the center one.

In accordance with an embodiment of the present invention, at least two diagnostic strips 14 are provided, enabling the detection of a plurality of indicators, and/or the detection of variable quantitative assessments of the same indicator, i.e. detectors with different cut-offs with regards to a positive or negative results, which depends on the quantity of the detected substance.

In some embodiments, the detection is directed towards Calprotectin, Lactofferin, S100A12, Pancreatic Elastase, as well as premier inflammatory and metabolic biomarkers such as EPX and Beta-glucaronidase, Anti-Gliadin-IgA-Antibodies, M2PK, sDNA, C. difficile bacteria, Helicobacter pylori, parasites, Candida, leucocytes, Rota virus, Chlamydia, Salmonella, PH.

In various embodiments, the inner components provided in housing 11 may be further isolated from one another with at least one elastomeric gasket 21, provided for sealing and securing, and which may be provided as an elastic O-ring.

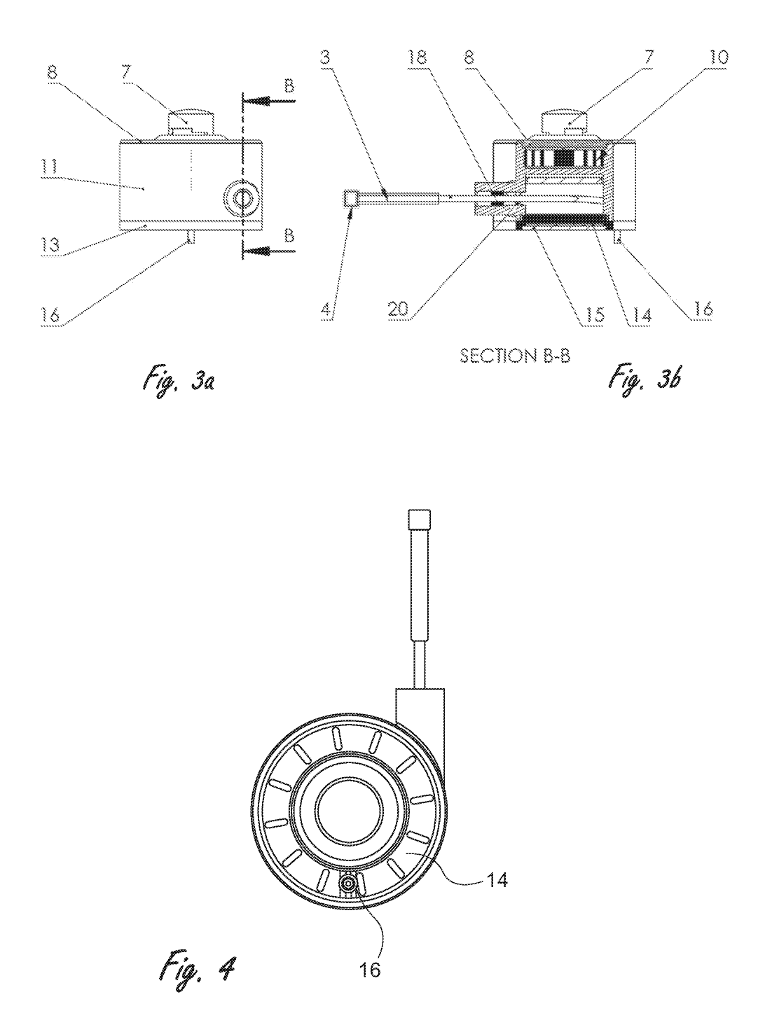

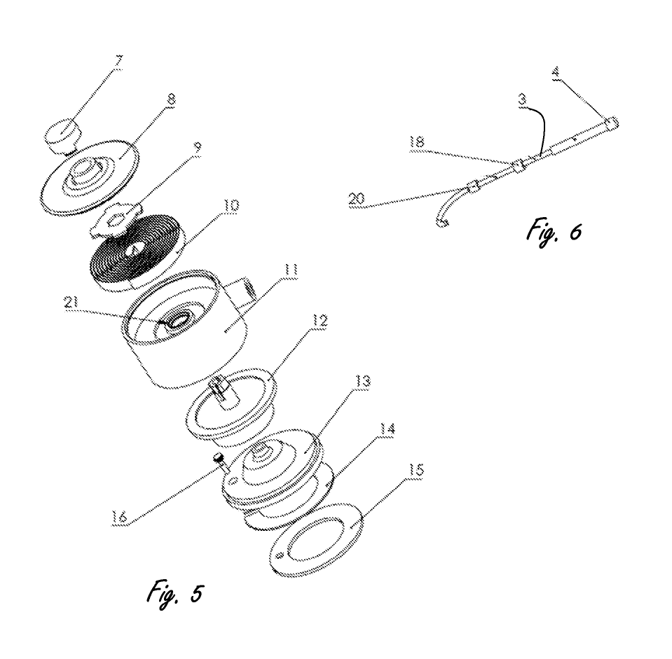

Reference is now made to FIGS. 3a and b schematically illustrating a side view and a cross-section of said side view, respectively, presenting the apparatus as shown in FIG. 2. FIG. 3b provides a cross-section view of the line B-B shown in the side view of FIG. 3a, and shows the inner workings of the swab-conducting tube 5, which in preferred embodiments is also provided with at least one resecting lip, 18 and 20.

Reference is now made to FIG. 4 schematically illustrating a bottom view of the embodiment of the apparatus as disclosed by the present invention, and showing the homogenization release member 16 which facilitates stool homogenized buffer flow into diagnostic strip 14.

Reference is now made to FIG. 5 schematically illustrating a three-dimensional explosive view of an embodiment of the apparatus of the present invention, as shown in FIG. 2. The operating mechanism can easily be shown through this illustration, showing that pressing operation button 7, leads through top cover 8 to the mechanical dislocation of release member 9, which releases the spring in retraction/spring-like mechanism 10, leading to the influx of stool containing swab into housing 11, through the swab-conducting tube. After passing bottom cover 12, the homogenized stool-buffer liquid may be leaked onto the diagnostic strip 14, after being released through sealing cover 13 and by switching homogenization release member 16 to the ON configuration. Diagnosis cover 15 is provided in a circular shape which is designed to cover and protect the circular diagnostic strip 14, and is preferably transparent to enable viewing of the diagnostic results.

Reference is now made to FIG. 6 schematically illustrating a three-dimensional perspective view of the retractable swab having various embodiments, provided as cap 14 and as resection lips 18 and 20, which may be provided as a one directional valve, a slit-like opening, a round-like opening, a duck-beak-like shape and any combination thereof, and may be located affixed in said housing, provided outside said housing, provided along said retractable swab and any combination thereof. In various embodiments, the resecting lips may be modified to allow passing of various sample sizes.

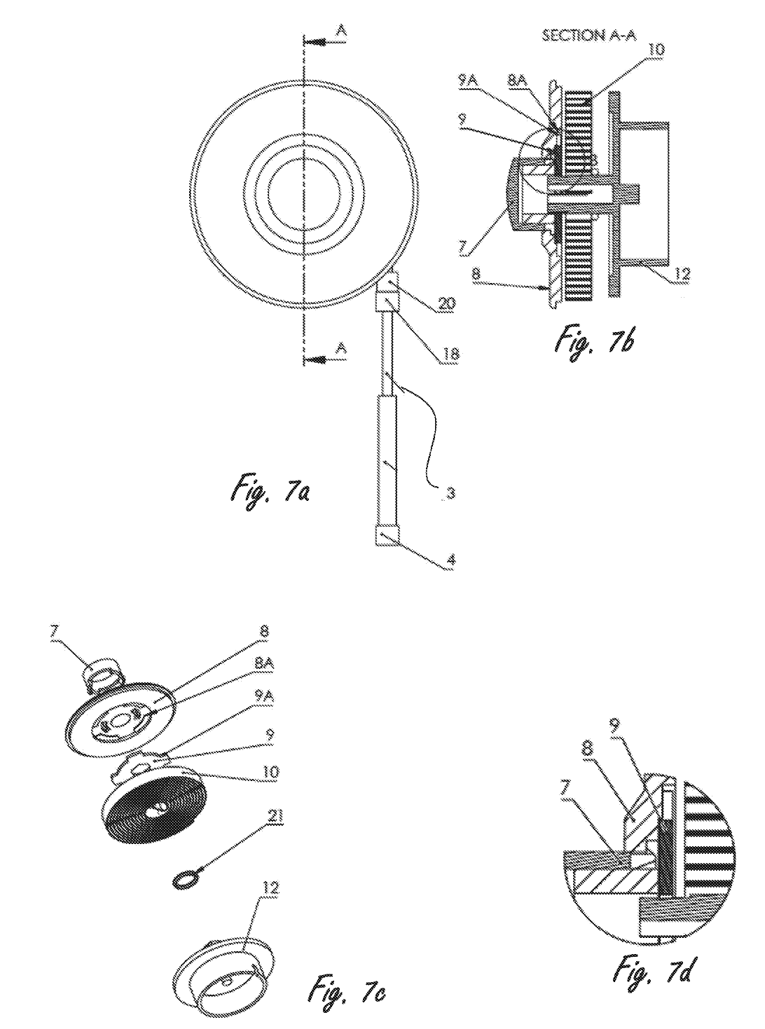

Reference is now made to FIGS. 7a-d, illustrating a top view, a side cross-section, an explosive three-dimensional view and a close-up view of an embodiment of the retraction/spring-like mechanism as provided in the present invention, wherein top cover 8 is provided with slits 8A, allowing the penetration of flanges in operation button 7 to reach the release member 9, and thus release the flanges 9A of release member 9 from their secured position. Having been connected to retraction/spring-like mechanism 10, in this embodiment provided as a spring, the release of flanges 9A and the resulting release of release member 9 enable the release of spring 10 and the operation of the apparatus into retracted mode.

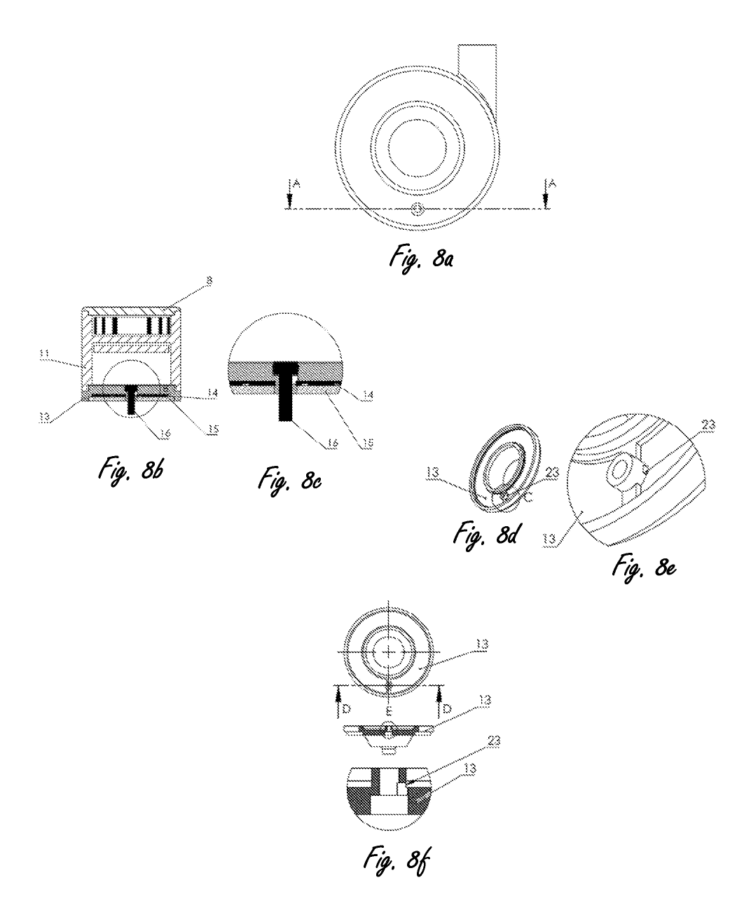

Reference is now made to FIGS. 8a-f, illustrating a bottom view, a side cross-section, a close-up cross-section, a perspective view and an explosive view of an embodiment of the present invention, respectively, having a release mechanism for allowing fluid communication between the chambers of the housing and the detector. In this embodiment, the release mechanism is provided by a slit 23 in sealing cover 13, which is incorporated with homogenization release member 16, which in its OFF configuration serves as a sealing member. Dislocating homogenization release member 16 essentially removes the obstacle of its action as a sealing member and provides fluid communication through slit 23.

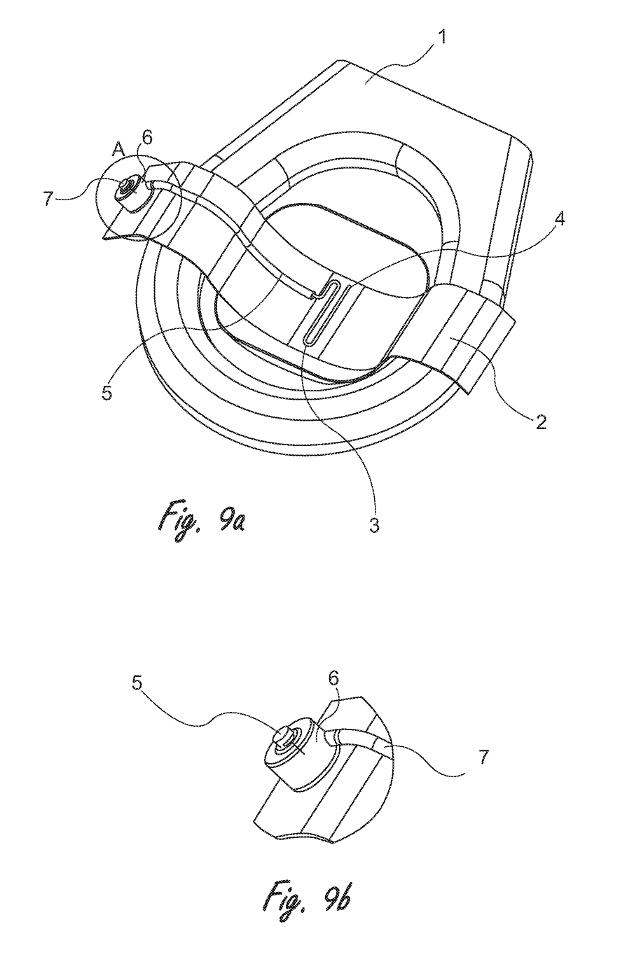

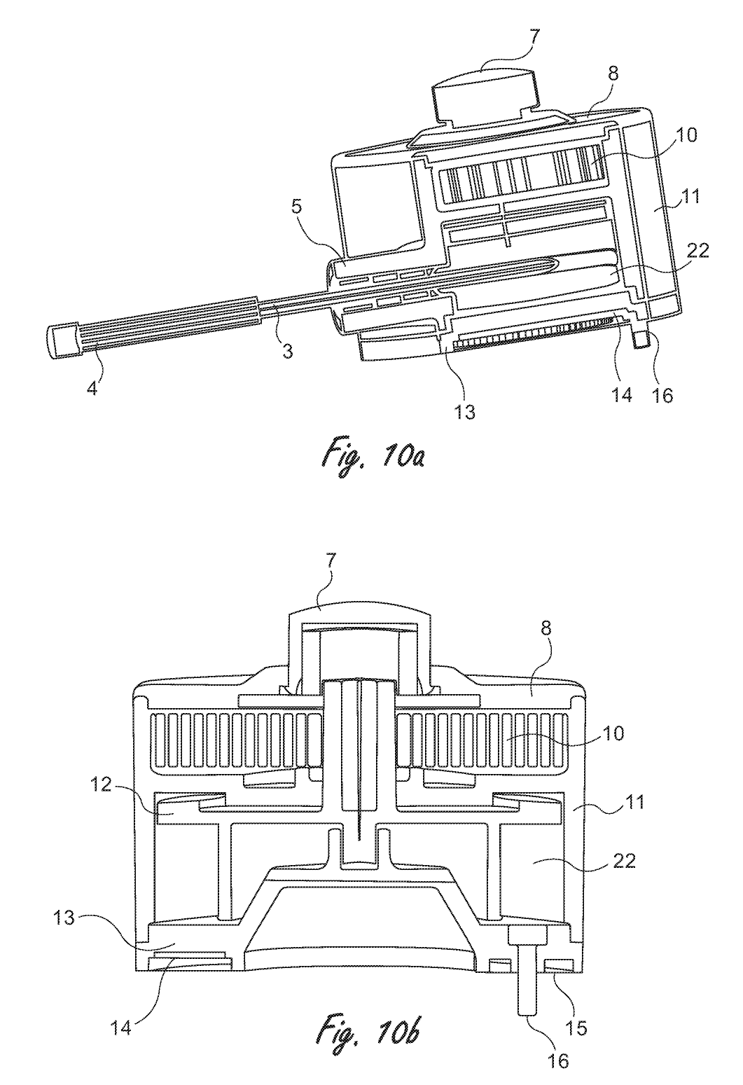

Reference is now made to FIGS. 9a and b schematically illustrating a perspective and a close-up views, respectively, of an embodiment of the present invention provided in the apparatus being connected to stool collector 2. The stool collector, which is further disclosed in FIGS. 11-18, is configured to be placed upon a toilet seat 1, and is further configured to have the apparatus of the present invention attached to it, and having the distal end of the retractable swab 3 accommodated in the location where stool is expected to be eliminated onto.

Thus, FIG. 9 exemplifies a possible use and application of the applicator provided by the present invention, wherein the user puts the stool collector 2 onto his toilet seat 1, comfortably provides a stool elimination onto the stool collector 2, where the distal end of retractable swab 3 is accommodated, and afterwards he may operate operation button 7 resulting in retracting swab 3 into housing 11 and thereby causing whirling of the stool with an extraction-homogenization buffer, resulting in homogenized stool in a single click.

FIGS. 10a and b illustrate two cross-section views of a three-dimensional perspective view of an embodiment of the apparatus as disclosed herein, exemplifying swab 3 retraction in FIG. 10a, and the homogenization chamber 22 in FIG. 10b, and wherein like components have like reference numbers as provided above.

Reference is now made to FIG. 11, illustrating in an out-of-scale a side view of the system 100 as disclosed by the present invention. The system is adapted to be used in a toilet 70, and is designed to collect stool samples into a container, providing stool which readily available for detection. The system comprises stool collector 110, which is adapted to be placed onto the toilet seat 40 for comfortable usage. Stool collector 110 is thus configured to be reversibly installed within a toilet-bowl, and is characterized by a nest-shaped structure comprising an upper opening, which is at least partially encircled by a toilet-attaching circumference, and a concave central portion, which is interconnected to the circumference, wherein the central portion is configured for confining stool's specimen. Positioned in the stool collector is retractable string-like swab 120, having a distal and proximal opposite ends, and configured to have its distal end positioned in the concave central portion of stool collector 110 where it is being made in contact with the stool specimen, while its proximal end is positioned outside of the concave central portion and is found in swab extender (housing) 130. Housing 130 is found in connection with the stool collector 110 and is preferably mechanically attached to the stool collector, as if hanging in proximity to toilet seat 40. Swab 120 is designed to have its proximal end positioned in housing 130, and is configured to be deployable in two alternative configurations, namely EXTENDED configuration and RETRACTED (_{collapsed}) configuration, whereby in the extended configuration, the swab distal end is located in stool collector 110 and in the retracted configuration, the swab is retracted from its collector location and into housing 130.

In preferred embodiments, housing 130 comprises container 134 having in it a buffer for homogenizing the stool sample which is collected by swab 120, and pumping/vacuum unit 132, adapted to operatively pull/push the stool or the buffer into the same compartment.

In most preferred embodiments, housing 130 further comprises at least one detector for identifying various characteristics of the stool collected, as disclosed in the present invention. The detector may be found within buffer container 134, and may be admixed with it prior to usage, just before usage or after usage of the stool collector. Thus, each detector means may be found in a separate container, found within the capacity of container 134, and mixing of each desired detector may be executed by operating unit 132.

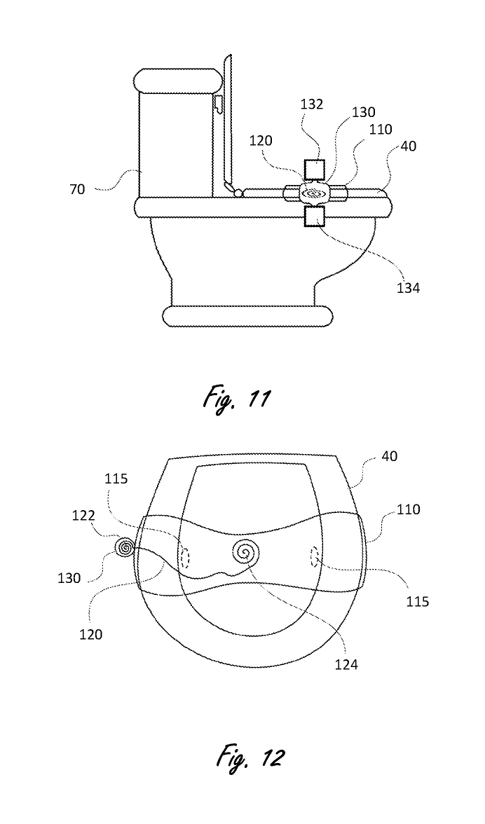

Reference is now made to FIG. 12, illustrating in an out-of-scale a top view of the system 100 as disclosed by the present invention, and illustrated in FIG. 11, and wherein like numbers represent like components. It can be seen that swab 120 is characterized by distal end 124 found in the concave portion of stool collector 110, and proximal end 122 found within housing 130.

FIG. 12 illustrates an embodiment of the present invention wherein stool collector 110 is characterized by a wide elongated shape, and being attached to toilet seat 40 with fastening means 115, which could be in a non-limiting example, water-miscible glue, or any other means available for attaching a sheet, such as Velcro attachments and the like.

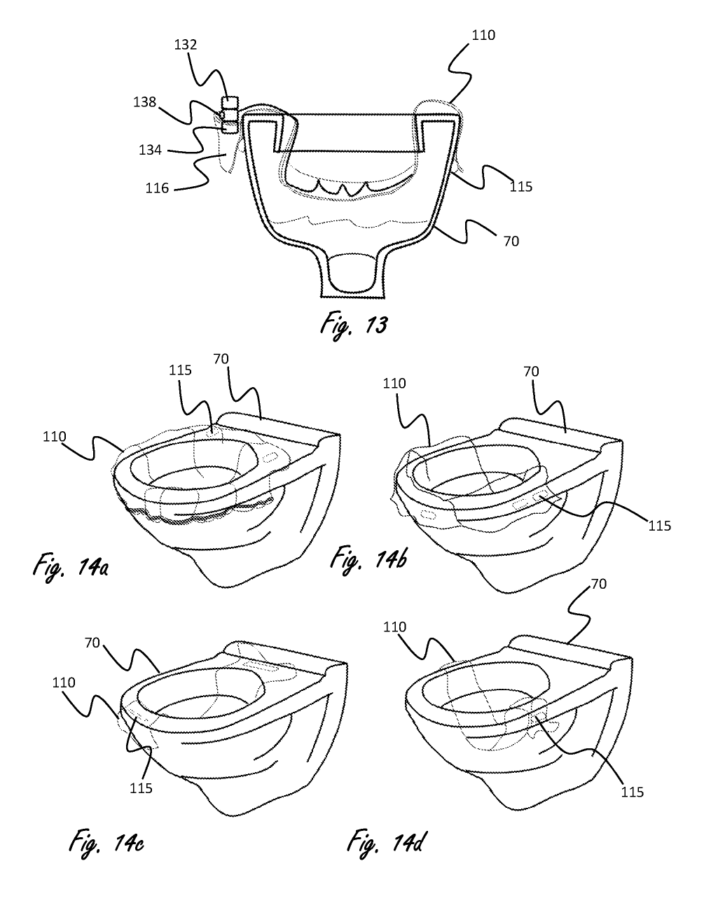

Reference is now made to FIG. 13 illustrating a cross-section of the system 100 as disclosed by an embodiment of the present invention, and showing an embodiment wherein stool collector 110 comprises an extension 116 adapted to cover housing 130;

One object of the present invention is to present a stool collector configured to be reversibly installed within a toilet-bowl 70. Reference is now made to FIG. 14a-d, 15a-c, 16, 17 and 18a-c disclosing a stool collector configured to be reversibly installed within a toilet-bowl, characterized by a nest-shaped structure comprising an upper opening, at least partially encircled by a toilet-attaching circumference, and a concave central portion, interconnected to said circumference, said central portion configured for confining stool's specimen.

Reference is now made to FIGS. 14a-d disclosing perspective illustrations of the stool collector as defined above, wherein the toilet-attaching circumference comprises one or more (a) elastic portions and/or (b) flaps 115, configured to fasten said collector, by means of (a) said elastic portions and/or (b) flaps, to the toilet's bowl, toilet seat or a combination thereof. It is well within the scope of the invention wherein toilet seat 40, temporarily, when used, fasten the flaps and firmly attaches the collector within the toilet bowl. Reference is now made to FIGS. 14a-d disclosing perspective illustrations and especially to FIG. 17 disclosing cross section of the stool collector as defined above, wherein said toilet-attaching circumference, along one or more portions thereof, comprises or otherwise provided in connection with a water-miscible glue, configured to fasten said collector, by means of said glue, to the toilet's bowl 70, toilet seat 40 or a combination thereof. A combination of both physical fastening and gluing flaps is well in the scope of the invention.

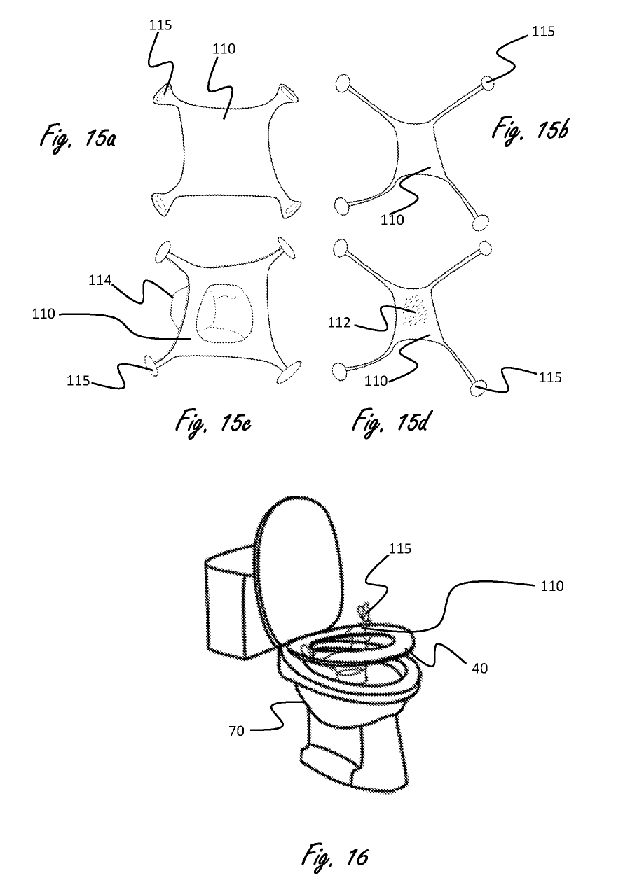

Reference is now made to FIG. 15c disclosing a perspective top view of the aforesaid stool collector which may further comprises a continuous envelope configured to connect said central portion to said toilet-attaching circumference in a continuous manner.

The present invention further discloses the aforesaid stool collector wherein at least a portion of said collector entirely obstructing the passage of light.

Reference is now made to FIGS. 15a, 15b and 15c disclosing perspective top views of the aforesaid stool collector wherein said collector further comprises a net-like perforated envelope configured to connect said central portion to said toilet-attaching circumference in a non-continuous manner.

Reference is now made to FIG. 15d disclosing perspective top view of the aforesaid stool collector wherein said collector further comprises in, on or in connection with said central portion stool specimen immobilizing means (SSIM). The SSIM may be selected form a group comprising of an abrasive material, an absorbing material, porosive material, layered and multi-laminar material, emulsion, liposomal, salt-containing material, buffer-containing material, aggregates and solid particales, fibers-containing matter, filament(s)-containing material, premoistened material, spiral-wound dressing and a combination thereof.

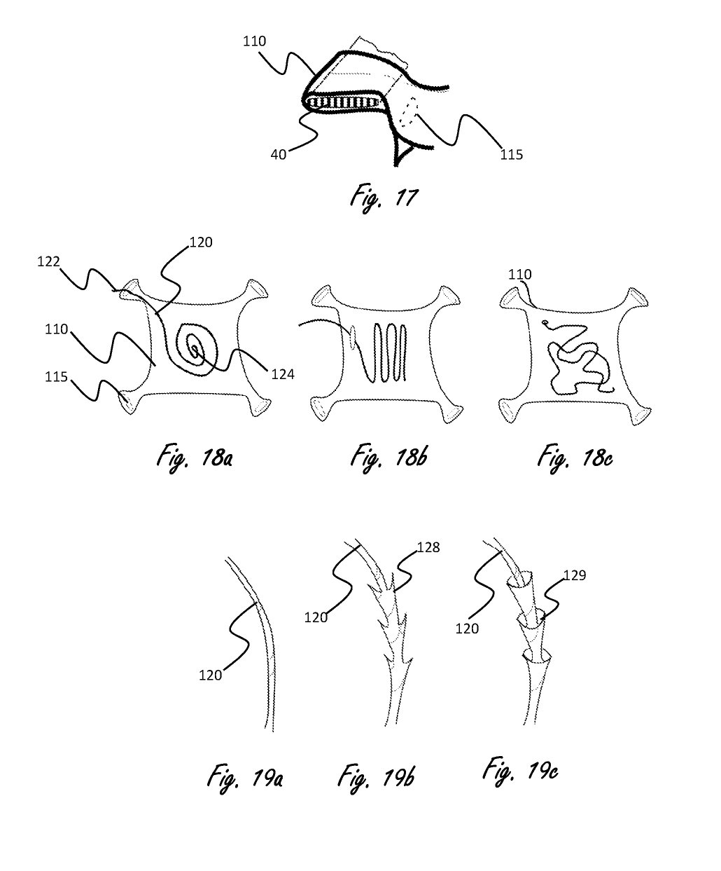

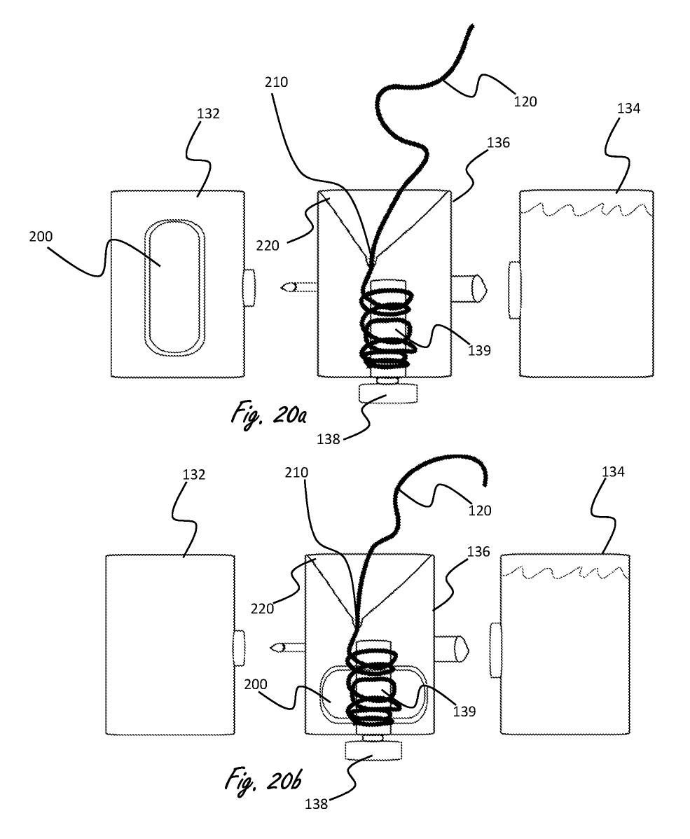

Reference is now made to FIG. 16 disclosing perspective illustration of stool collector 110 as defined above, wherein the toilet-attaching circumference, along one or more portions thereof, comprises or otherwise provided in connection with a rope 115, configured to fasten said collector, by means of said rope, to the toilet's bowl 70, toilet seat 40 or a combination thereof.

Reference is now made to FIG. 18a-c and FIG. 15a-d disclosing top views of various SSIMs 115. More than that, the present invention further discloses the aforesaid stool collector 110 wherein said SSIM is selected form a group consisting of an abrasive material, an absorbing material, porosive material (FIG. 15), layered and multi-laminar material, emulsion, liposomal, salt-containing material, buffer-containing material, aggregates and solid particles, fibers-containing matter, filament(s)-containing material (FIG. 18), pre-moistened material, spiral-wound dressing and any combination thereof.

The present invention further discloses the aforesaid stool collector 110 wherein it is at least partially made of one or more members of a group consisting of pre-moistened material, flushable towelette, flushable towelette impregnated with polyvinyl alcohol-containing binders, non-woven web and a patterned sheet thereof, toilet paper and the like, toilet tissues, tissues, wipes in various grades, a paper-made patterned sheet, polymeric material, especially polylactic and polyglutamic acid-containing polymers, polyamide-containing polymers and any combination thereof.

The present invention further discloses the aforesaid stool collector 110 wherein said collector further comprises a pierceable surface for collecting a stool specimen form said central portion.

The present invention further discloses the aforesaid stool collector 110 wherein said collector further comprises a detector for testing said stool specimen.

The present invention further a closeable stool collector which is, further characterized by an OPEN configuration where the collector is attached to the toilet with its upper opening being accessible, and a CLOSE configuration where the collector, by means of said circumference or attachment thereof, is tightly sealed such that stool is securely contained within.

Reference is now made to FIG. 19A-C schematically illustrating suggested embodiments of retractable swab 120 shape and form, as disclosed by the present invention, and illustrating in FIG. 19a a smooth rope, thread or the like, and in FIGS. 19b and 19c projections 128 and 129, respectively, adapted to more efficiently collect stool onto swab 120. It is a novel feature of the present invention, wherein swab 120 is retracted directly from stool collector 110, straight into housing 130, which in preferred embodiments contains a homogenizing buffer and in most preferred embodiments, also comprises at least one detector.

Reference is now made to FIG. 20A-B illustrating in an out-of-scale a side view of two suggested embodiments of the swab extender (housing) 130; Swab 120 is adapted to be incorporated in housing 130 with its proximal end 122, while distal end is found away from housing 130 in its extended configuration and inside housing 130 in its retracted configuration. In order to effectively retract swab 120 into housing 130, embodiments of the present invention provide swab wrapping, or winding means 139. In some embodiments of the present invention, winding means 139 are operatively winded to allow winding or unwinding of swab 120, to enable its extension or retraction. Such winding may be operated by operating switch 138, which may operate having a mechanical spring, an electrical motor, pneumatic motor and the like. Housing 130 main compartment 136, for containing swab 120, may be supplied with cup 220, having a funnel-like shape, for containing swab 120 and any possible spatters when transitioning between extended and retracted configurations. At the proximal end of cup 220, having the narrowest aperture, a preferred embodiment provides stool resecting lips (SRLs) 210, touching at least one surface of said swab, and configured to resect surplus stool attached to said swab when deployed from its EXTENDED configuration to its RETRACTED (_{collapsed}) configuration, thereby providing meter-dose sample within said housing.

In some embodiments of SRLs 210, they may be provided in a shape selected from a group consisting of a one directional valve, a slit-like opening, a round-like opening, a duck-beak-like shape and any combination thereof, and in preferred embodiments they may be configured to enable modification of their resecting sample size, providing a predetermined meter-dosed sample in a plurality of sample sizes which may enter into the inner portion of housing 130.

In preferred embodiments of the present invention, housing 130 further comprises at least one container 134 provided with at least one buffer solution. The buffer is intended for homogenizing the stool sample, and/or for containing detection means. In various embodiments the container may be split into at least two sub-containers, each of which contains a different buffer or detection solution, and the sub-containers are adapted to homogenize and merge when operating housing 130 with a pump/vacuum unit 132. Unit 132 is also configured to allow suction of the buffer solution through the stool, or vice versa, providing homogenization of the stool with the buffer solution. A membrane may also be provided to selectively allow only buffer solution after being in contact with the stool, and being transferred to unit 132, which may comprise detection window 200. Containers 132 and 134 may be modular in nature, and the manufacturer or the end user may be allowed to switch between various containers and detectors available in individual wrappings. FIG. 20a and FIG. 20b illustrate alternative positions for the detection window 200, providing two embodiments of detection reaction location.

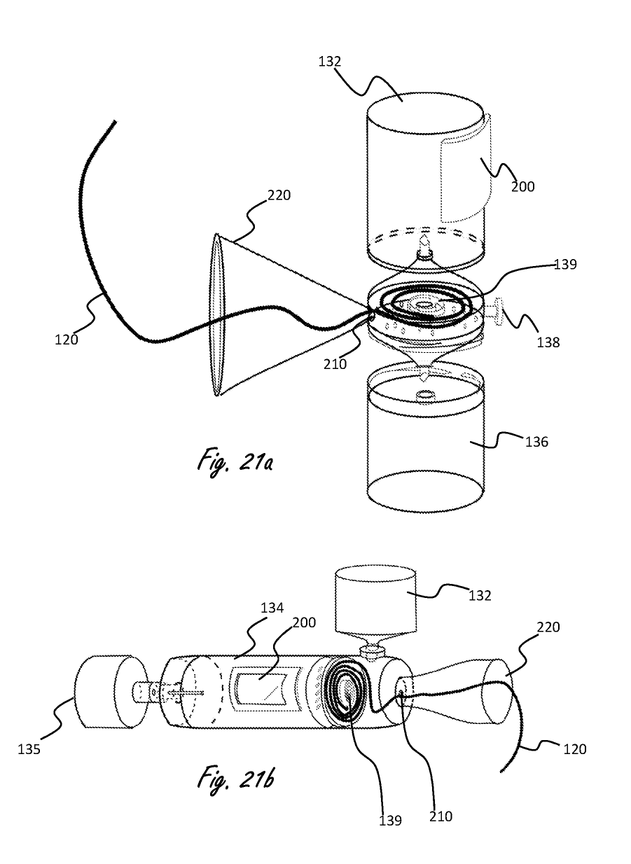

Reference is now made to FIG. 21a-b illustrating perspective out-of-scale view of another two suggested embodiments of the swab extender (housing) 130, in which like reference numbers represent like components. FIG. 21a represents an alternative wherein cup 220 is located outside of housing 130, and rotational motor 139 enables rotational collection of swab 120, through operating switch 138. FIG. 21b represents an alternative configuration of the containers, positioning container 132 perpendicular to the main axis of housing 130, and adapting it to contain a detection solution or a second buffer than the buffer found in container 134. Unit 135 represents vacuum or pumping means, adapted to operate the flux of the solution in 132, through swab 120 and finally into container 134, where detection window 200 is available.

Another object of the present invention is to disclose a retractable stool's specimen swab, as schematically illustrated in FIGS. 18, 19, 20 and 21 schematically illustrating a retractable stool's specimen swab 120, which is characterized by an endless thin, string-like, stool's swab 120 having a distal 124 and proximal 122 opposite ends, said proximal end is attached to a swab′ extender 130 (_{housing}) which may comprise one or windows or analysis displaying means 200 and stool's sample dosing element (lips 210 for resecting surplus stool from swab); said distal end is deployable in two alternative configurations, namely EXTENDED configuration and RETRACTED (_{collapsed}) configuration. The swab's distal end is in connection with a swab's pulling/pushing mechanism (SPPM, 132) configured to deploy said distal portion 124 from said EXTENDED to RETRACTED and/or vice versa. SPPM possibly provided in connection with one or more buffer-homogenizing members or wings.

The present invention further discloses self-retracting stool's specimen swab as defined above, wherein said swab made of or otherwise comprising at least one material selected from a group consisting of an abrasive material, an absorbing material, porosive material, layered and multi-laminar material, emulsified material, liposomal material, salt-containing material, buffer-containing material, aggregated material, solid particles, fibers-containing material, filament(s)-containing material, pre-moistened material and any combination thereof.

In another embodiment of the invention, a self-retracting stool's specimen swab is disclosed wherein in said RETRACTED configuration, said swab is spirally-wounded.

In another embodiment of the invention, SPPM comprises one or more members of a group consisting of a spring, a vacuum pump, a pneumatic motor, an electric motor, a manual motor and any combination thereof.

In another embodiment of the invention, the vacuum pump is a vacutainer, such as the commercially available blood collection tubes which are sterile glasses or plastic tubes with a closure that is evacuated to create a vacuum inside the tubes facilitating the draw of a predetermined volume of liquid.

In another embodiment of the invention, the swab′ extender (_{housing}) further comprising a detector for testing said stool specimen. In another embodiment of the invention, swab′ extender (_{housing}) further comprising a predefined volume of a buffer, for diluting said sampled stool specimen. In another embodiment of the invention, swab′ extender (_{housing}) further comprising at least one detector for testing said stool specimen. In another embodiment of the invention, the swab′ extender (_{housing}) further comprising a predefined volume of a buffer, for diluting said sampled stool specimen, and at least one detector for testing said buffer-diluted stool specimen.

In another embodiment of the invention, at least a portion of said swab′ extender (_{housing}) is transparent such that said stool's test, detect by said detector, is visible outside said housing.

In another embodiment of the invention, the detector is barriered from said buffer-diluted stool by means of a separating membrane which is configured to selectively facilitate buffer's influx while it effectively blocks stool contact with said detector.

Another object of the present invention is to present a kit for collecting, sampling and testing stool specimen. According to an embodiment of the invention, the aforesaid kit comprises a stool collector configured to be reversibly installed within a toilet-bowl, characterized by a nest-shaped structure comprising an upper opening, at least partially encircled by a toilet-attaching circumference, and a concave central portion, interconnected to said circumference, said central portion configured for confining stool's specimen; and one member of a group consisting of (a) a stool sampler, characterized by an elongated syringe-shaped structure characterized by an open bore needle, having an applicable volume (Vn), at distal end, for piercing a pierceable surface of a stool-containing collector, thereby extracting a metered dose (Vs) stool's specimen, Vn equals Vs; and an elongated open-bored body, in a fluid connection with said needle via a needle-inlet provided in said body's envelope, comprising a predefined volume confined in said envelope, said body contains a predefined volume of a buffer, for diluting said sampled stool; and at least one detector for testing said buffer-admixed stool specimen; (b) a retractable stool's specimen swab, characterized by an endless thin stool's swab having a distal and proximal opposite ends, said proximal end is attached to a swab′ extender (_{housing}); said distal end is deployable in two alternative configurations, namely EXTENDED configuration and RETRACTED (_{collapsed}) configuration; and (c) a stool testing retractable swab, characterized by said endless thin stool's swab, wherein said swab′ extender (_{housing}) further comprising a detector for testing said stool specimen.

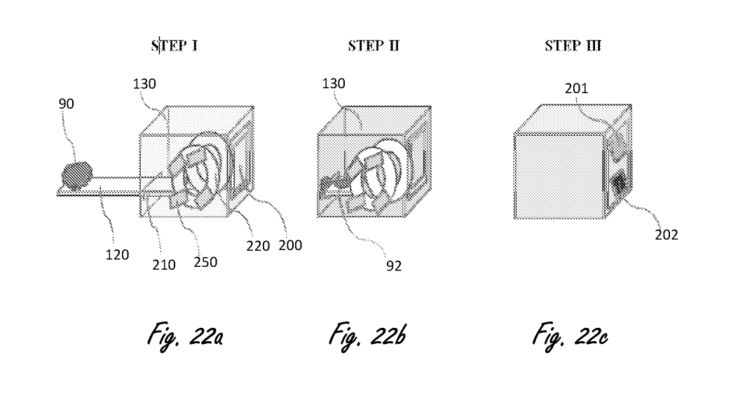

Reference is now made to FIG. 22 schematically illustrating an exemplified use of the extractable swab 120 and swab extender 130 having two detectors 201 and 202. Step I (FIG. 22a) depicts sampling stool 90 in liquid form, solid form or semi-solid (gel) by a retractable swab 120. The swab dimensions are configured to provide the analysis volume a specific, predefined and constant (meter-dose) stool analysis volume. Stool resecting lips 210 can be used. The swab retracting mechanism 139 can be in connection with a plurality of swiveling, actuating or rotating wings which homogenize the stool-in-buffer solution so a homogeneous solution or suspension is provided. The device may also contain an analysis window 250 or any display for showing in a direct or indirect (e.g., wireless) manner. Step II (FIG. 22b) depicts the inflowing of meter-dose sample of stool on retracting swab to the analysis chamber whilst homogenizing the buffer with stool. In this step analysis is provided. Step III (FIG. 22c) depicts indicating analysis result(s) on one or more windows 250 showing, qualitatively or quantitatively, presence or one or more predefined suspected pathologies. In a manner of example, faecal calprotectin analysis is set for upper limit of 112 μg/g of faece; positive (green, upper window 201) indicates patient has lower calprotectin than set limit. Hence, patient apperantly has no IBD-related indication. The lactoferrin concentration (microgrammes/gram faecal weight) is 0±1.4 for IBS patients, 6.6±42 for UC patients, 4±12.7 for CD patients and 0.5±2 for healthy controls (median±interquartile range), see http://onlinelibrary.wiley.com/doi/10.1111/j.1365-2036.2010.04306.x/abstract, currently available link which is incorporated herein as a reference. Hence, simultaneously and concurrently to said calprotectin analysis, lactoferrin is analyzed in an analysis set at upper limit of 20 μg/g showing the patient has an idiopathic non-IBD infection. Thus, overall checkup of an IBD-patient shows he/she currently suffers from a GI infection which seemingly not related with his/her IBD illness. The patient is indicatively not within an IBD-acute stage, and will not be currently treated with steroids of biological drugs, but will be treated as if he/she as regular GI infection.

Another object of the present invention is to present a method of stool sampling, characterized by step of providing an elongated syringe-shaped structure with an open bore needle, having an applicable volume (Vn), at distal end, for piercing a pierceable surface of a stool-containing collector, thereby extracting a metered dose (Vs) stool's specimen, Vn equals Vs; and further providing an elongated open-bored body in a fluid connection with said needle via a needle-inlet provided in said body's envelope, comprising a predefined volume confined in said envelope; containing, in said body, a predefined volume of a buffer, thereby diluting said sampled stool; and further providing the same with at least one detector thereby testing said buffer-admixed stool specimen.

In another embodiment of the invention, the method defined above further comprising a step providing at least a portion of said envelope to be transparent such that said stool's test, detect by said detector, is visible outside said sampler.

In another embodiment of the invention, the method defined above further comprising a step separating, by means of a membrane, said buffer-diluted stool, and configuring the same to selectively facilitating buffer's influx while effectively blocking stool contact with said detector.

In another embodiment of the invention, the method defined above further comprising step of providing said sampler with a pumping mechanism and locating the same at said sampler's proximal side.

In another embodiment of the invention, the method defined above further comprising step of configuring said pumping mechanism to provide said body with an effective vacuum to influx said metered dose (Vs) stool's specimen, temporarily accommodated within the pierced needle, and further admix it with the buffer, such that homogenized buffer-diluted stool suspension is obtained.

In another embodiment of the invention, the method defined above further comprising step of reciprocally moving said needle along sampler's main longitudinal axis; configuring said needle, and sampled stool within, to sealably enter said sampler body via said needle-inlet whilst effectively blocking buffer's efflux.

In another embodiment of the invention, the method defined above further comprising step of configuring said pumping mechanism to provide said body with an effective vacuum thereby reciprocally sucking said needle, and sampled stool within, along sampler's main longitudinal axis into said body.

In another embodiment of the invention, the method defined above further comprising step of configuring said pumping mechanism to provide said body with an effective vacuum to withdraw said stool specimen out from said needle bore, and then (b) admixing said stool with said buffer thereby homogenizing the obtained stool-in-diluent suspension.

In another embodiment of the invention, the method defined above further comprising step of configuring said pumping mechanism to provide effective vacuum to thereby facilitating efflux of said buffer via said membrane towards said detector.

Another object of the present invention is to present a method of stool sampling by means of a retractable stool's specimen swab, comprising steps of providing an endless thin stool's swab with a distal and proximal opposite ends, attaching said proximal end to a swab′ extender (_{housing}); and deploying said distal end in two alternative configurations, namely an EXTENDED configuration and a RETRACTED (_{collapsed}) configuration.

According to this embodiment of the invention, the method defined above further comprising steps of connecting said swab's distal end with a swab's pulling/pushing mechanism (SPPM) and deploying said distal portion from said an EXTENDED configuration to a RETRACTED configuration and/or vice versa.

According to another embodiment of the invention, the method defined above further comprising steps of configuring at least a portion of said swab's distal portion to a shape, and selecting the same from a group consisting of cord-like thick and/or hairy fiber, dental-floss-like filament having a thin and narrow cross section; measuring tape-like swab having relatively wide and thin cross section and any combination thereof.

According to another embodiment of the invention, the method defined above further comprising step of making at least a portion of said swab from at least one material selected from a group consisting of an abrasive material, an absorbing material, porosive material, layered and multi-laminar material, emulsified material, liposomal material, salt-containing material, buffer-containing material, aggregated material, solid particles, fibers-containing material, filament(s)-containing material, pre-moistened material and any combination thereof.

According to another embodiment of the invention, the method defined above further comprising step of spirally-wounding said swab in said RETRACTED configuration.

According to another embodiment of the invention, the method defined above further comprising step of providing said SPPM with one or more members of a group consisting of a spring, a vacuum pump, a pneumatic motor, an electric motor, a manual motor and any combination thereof.

According to another embodiment of the invention, the method defined above further comprising steps of providing said swab′ extender (_{housing}) with a detector and testing said stool specimen.

According to another embodiment of the invention, the method defined above further comprising step of providing at least a portion of said swab′ extender (_{housing}) to be transparent such that said stool's test, detect by said detector, is visible outside said housing.

According to another embodiment of the invention, the method defined above further comprising step of providing said swab′ extender (_{housing}) with a predefined volume of a buffer, thereby diluting said sampled stool specimen.

According to another embodiment of the invention, the method defined above further comprising step of providing said swab′ extender (_{housing}) with at least one detector for testing said stool specimen.

According to another embodiment of the invention, the method defined above further comprising step of providing said swab′ extender (_{housing}) with a predefined volume of a buffer, diluting said sampled stool specimen, and by means of at least one detector, testing said buffer-diluted stool specimen.

According to another embodiment of the invention, the method defined above further comprising step of separating said buffer-diluted stool by means of a membrane, thereby selectively facilitating buffer's influx while effectively blocking stool contact with said detector.

According to another embodiment of the invention, the method defined above further comprising step of providing said extender (_{housing}) with one or more stool resecting lips (SRLs); affixing said SRLs in said housing to touch at least one surface of said swab, and resecting surplus stool attached to said swab when deploying the same from its EXTENDED configuration to its RETRACTED (_{collapsed}) configuration, thereby providing meter-dose sample within said housing.

Another object of the present invention is to disclose a method of stool collecting, sampling, and testing, comprising steps of providing a stool collector to a nest-shaped structure comprising an upper opening, at least partially encircling it by a toilet-attaching circumference, and providing it with a concave central portion, interconnecting the same to said circumference, configuring said central portion confining stool's specimen; and then reversibly installing a stool collector within a toilet-bowl, until stool sample is collected; and one member of a group consisting of (a) shaping a stool sampler to elongated syringe structure and providing it with an open bore needle, having an applicable volume (Vn), at distal end, for piercing a pierceable surface of a stool-containing collector, extracting a metered dose (Vs) stool's specimen, Vn equals Vs; and an elongated open-bored body, providing said needle, via a needle-inlet, in a fluid connection with body's envelope, confining a predefined volume in said envelope, containing in said body a predefined volume of a buffer, diluting said sampled stool; and by means of at least one detector, testing said buffer-admixed stool specimen; (b) providing a retractable stool's specimen swab with an endless thin stool's swab member having a distal and proximal opposite ends, attaching said proximal end to a swab′ extender (_{housing}); deploying said distal end in two alternative configurations, namely an EXTENDED configuration and a RETRACTED (_{collapsed}) configuration; and (c) providing a stool testing retractable swab, with an endless thin stool's swab member, providing said swab′ extender (_{housing}) with a detector, and testing said stool specimen by mean of said detector.

Another object of the present invention is to disclose a method of diagnosing IBD, especially CD and UC, GI disorders, infections, inflammations, and trauma, FIT, sDNA, FOBT, gFOBT, FOB, fecal lactoferrin (Lf), calprotectin (Cal), polymorphonuclear neutrophil elastase (PMN-e), as well as serum C-reactive protein (CRP).

It is in accordance with an embodiment of the present invention to provide the retractable swab apparatus with an imaging means, such as a camera. Such a camera may be an embedded camera feature in an electronic device, such as a mobile phone or a tablet. The camera is configured to enable imaging of either a stool specimen after being retracted into the housing, or the detection unit, after being in contact with homogenized/extracted stool. According to this embodiment, a processor is also provided, which is configured to read software instructions for obtaining the images from the imaging means, processing them and optionally sending the raw images, or the processed results to a second electronic device. Such an electronic device could be a server, a mobile phone, a tablet, a personal computer or a cloud-like server, and it could be used by medical personnel, which would be able to provide feedback to the communicated data. Processing of the images may result in estimated parameters relating to the raw stool specimen or the finalized detection results, and may be, in a non-limiting example, a detection of stool specimen color spectrum, detection of stool consistency, detection of a positive or negative result in the detection unit, quantitative results of the intensity of the results in the detection unit, and the like. Such results enable detection of various conditions, such as occult blood in stool, presence of infections, or presence and quantification of various drugs and medications.

### Example 1

One embodiment of the aforesaid retractable stool's specimen swab is hereby presented as a non limiting example. This stool sample collecting and diagnosing device and kit thereof is characterized by a first (e.g., multiuse-) module comprising a motor actuating a rotatable axle; and a second (e.g., disposable-) module comprising: a rotatable shaft reversibly connectable with the axle, the shaft is further connected to and wounded by an elongated cord's proximal portion, the cord's distal portion comprises means for retaining stool material when introduced in or at the surface of stool to be sampled; a pierceable chamber with predefined volume of stool-sample homogenizing fluid; a stool-sampling cabinet of a predefined volume throughout which the cord is crossing from the shaft via at least one stool resecting lip and from an aperture of the cabinet cork outwardly, the cabinet configured to accommodate a meter dosed stool sample; and a diagnosing strip to which a predefined small volume of stool sample is introduced after being wet by the homogenizing fluid.

It is another object to of the present invention to provide a method of stool sample collecting and diagnosing by means of a device as defined above and below.

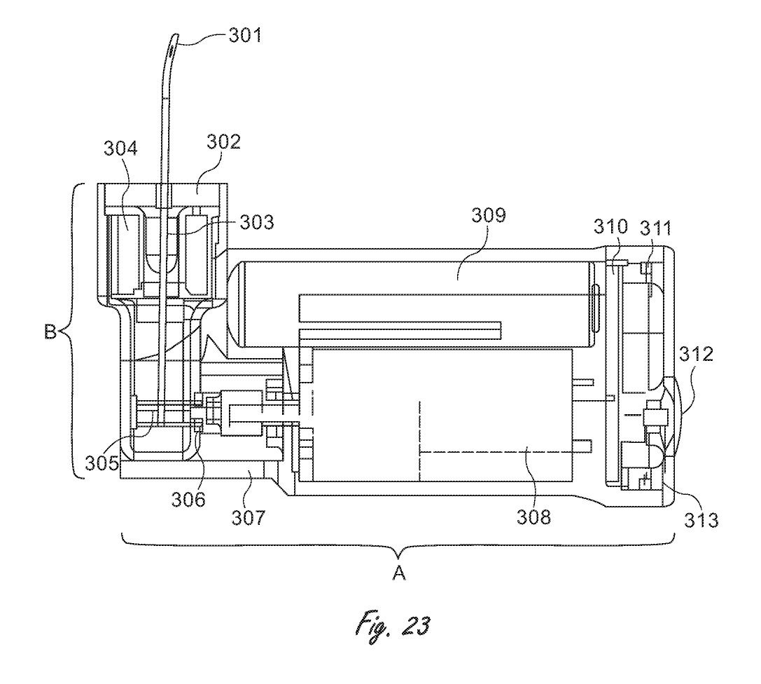

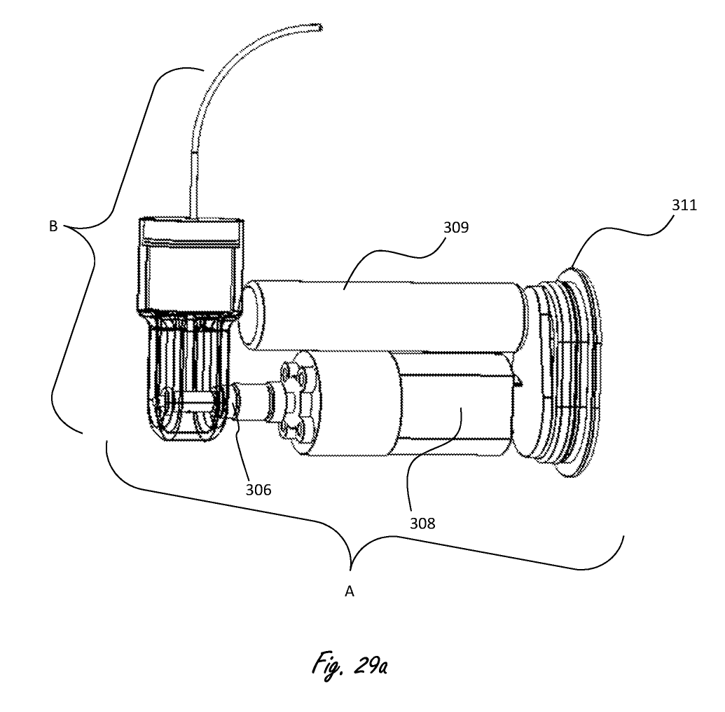

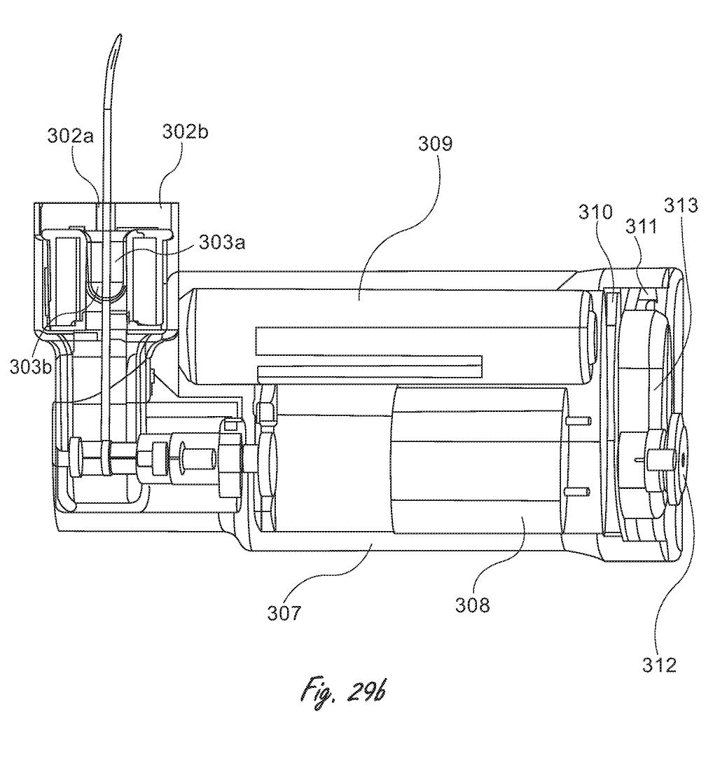

Reference is now made to both FIG. 23 & FIG. 29a&b, which schematically illustrate a side view of a stool sampler according to an embodiment of the present invention. This stool sampler, interchangeably further termed hereinafter as “device”, “sampler”, “kit”, comprises as a kit two modules: a first module, provided hereinafter in a non-limiting manner as a multiuse module, A comprising inter alia a motor; and second module, provided hereinafter in a non-limiting manner as a disposable, changeable or replicable module, B, reversibly interconnectable to said first (e.g., multiuse-) module, A and comprises the collecting and diagnosing systems.

Let's begin the description with the description of one embodiment of the disposable module, B. An elongated stool sampling cord (301), having a main longitudinal axis, the cord comprises a stool retaining structure and surface at its distal portion, and a proximal tip configured to anchor a rotatable shaft (305) thus reciprocally and reversibly be wounded along shaft (305), thereby elongating or shortening cord's length along its main axis to a required measure.

In this embodiment, the cord snugly pierces an aperture (302a) in a cork or stopper (2b) plugged over both (i) an inner meter-dozed stool sample collecting cabinet (303a) and (ii) a tube-like container (304) provided in a concentered manner outwardly said cabinet (303a). Stool sample collecting cabinet (303a) is having at least one stool resecting lip, here, e.g., a syncline of concaved rubbed lips (303b), wherein container (304) is configured to retain a predefined measure of a fluid for homogenizing said meter-dosed stool-sample.

As defined above, rotatable shaft (305) is in connection (e.g., coupled directly or provided via a gear mechanism) with an axle of electric motor (308) that spins the interconnected cord clockwise, counter-clockwise or both, in one speed (single RPM) or in a range of RPMs. It is in the scope of the invention when the motor is selected from various types possible, e.g., electric motors, mechanical motors, spring loaded mechanical motors, automatic motors, semiautomatic or manually activated motors etc. By aforesaid shaft's rotation, the cord initially elongates to a desired length so the cord can be conveniently inserted to or adjacent to a stool to be sampled, e.g., stools in a remote toilet bowl. After cord's distal portion is contacted with said stool, shaft's rotation shortens the cord, and by its way backwardly, stool enters cabinet (303a) via cork's piercing aperture (302a), whist stool accommodates within stool cabinet (303a) and not leaving the cabinet due to the effective resection action and the wiping of the cord by resecting lips (303b). Hence, backward rotation of the shaft provided a meter-dosed stool sample within the sampling cabinet. Upon subsequent piercing of both cabinet (303a) and fluid container (304), fluid is effectively wetting the stool thus extracting the markers to be analyzed and provide the suspension to appropriate rheological properties allowing the sample to flow and be diagnosed on a diagnosing strip. Then, rapid rotation of the shaft spins cord's abutment, the effective stiffing homogenizing the wetted stool suspension, allowing predefined one or more soluble markers to wet one or more diagnosing strips, and thus quantitatively, semi-quantitatively or qualitatively real-time indicating the condition of a patient.

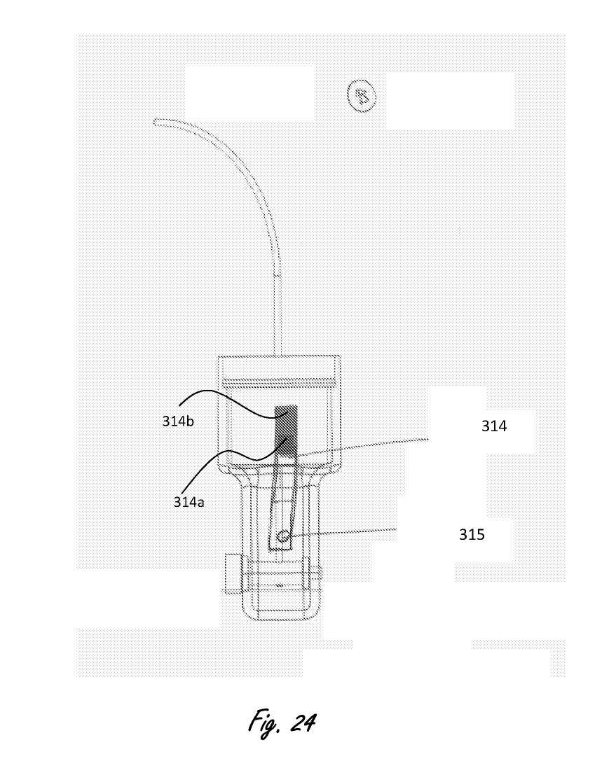

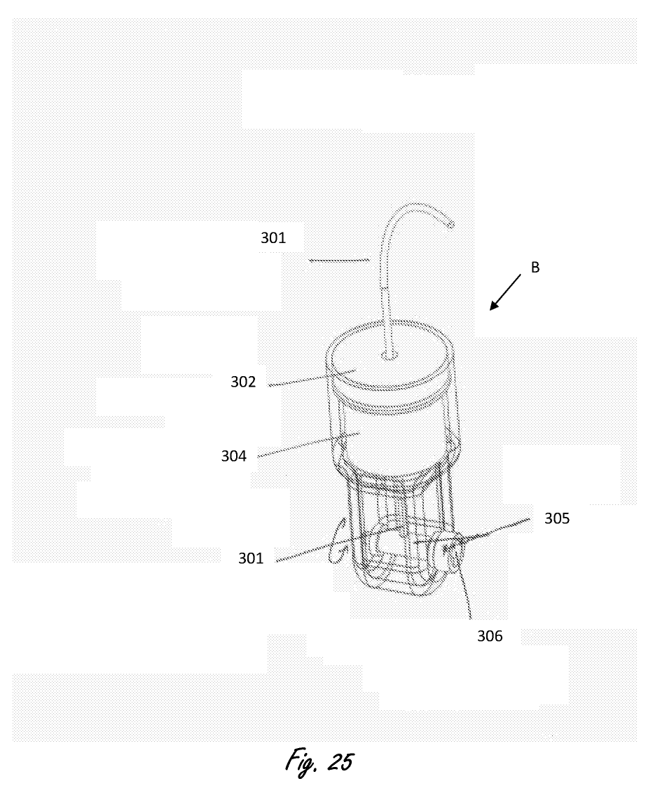

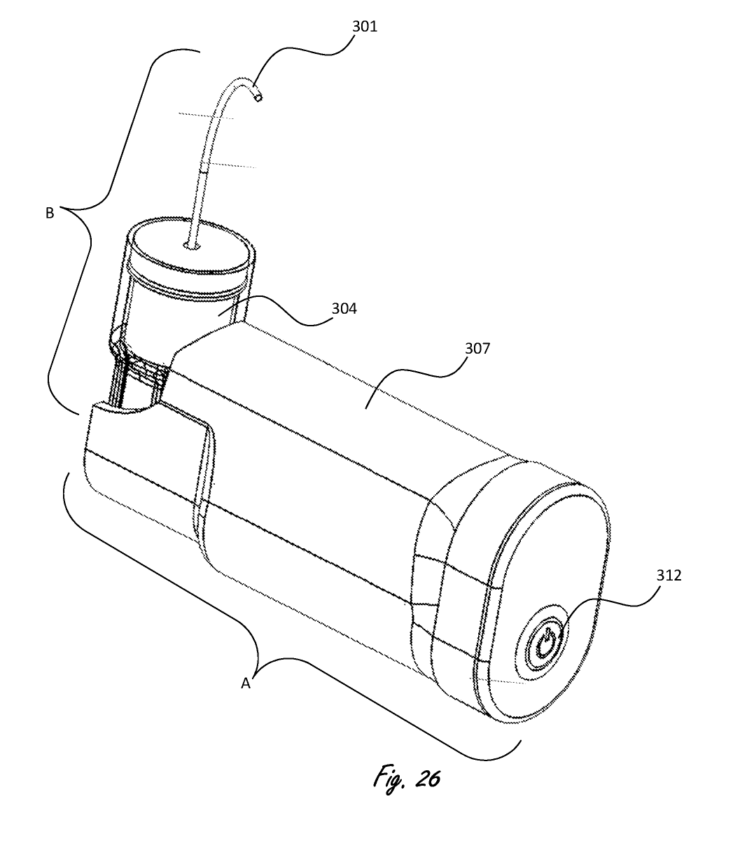

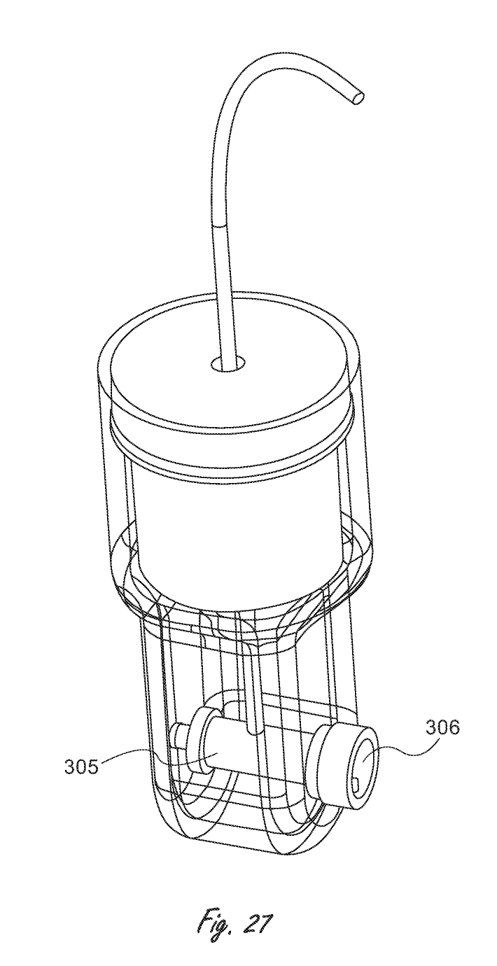

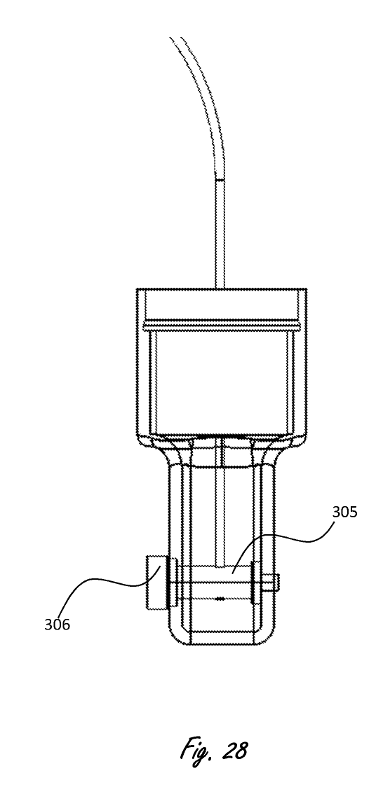

Reference is now made to both FIGS. 24-27 which schematically illustrate views of disposable module B of a stool sampler according to the above defined embodiment. FIG. 24 schematically shows one diagnostic strip (314), having, according to one embodiment of the technology red (314a) or green (314b) indications for under a critical measure or significant over critical measure of a marker of a medical condition, respectively; or according to yet another embodiment of the technology, indication of a first marker of a first condition (marking strip of window 314a) and/or second marker of a second condition (marking strip of window 314b) etc.

Hence for example, 60 μg/g faecal calprotectin can be provided as a critical measure for IBD condition, See Smith, Lyn A., and Daniel R. Gaya. “Utility of faecal calprotectin analysis in adult inflammatory bowel disease.” World J Gastroenterol 18.46 (2012): 6782-678 which is incorporated herein with its citations as a reference. Much similarly, critical measures are indicative for fecal occluded blood tests, see Beg, M., et al. “Occult gastro-intestinal bleeding: detection, interpretation, and evaluation.” JIACM 3.2 (2002): 153-158; lactoferrin, see Vaishnavi, Chetana, Deepak K. Bhasin, and Kartar Singh. “Fecal lactoferrin assay as a cost-effective tool for intestinal inflammation.” The American journal of gastroenterology 95.10 (2000): 3002; those two and citations thereof are incorporated herein as references.

Reference is still made to FIG. 24 schematically illustrating a front view wherein diagnosing button (315) comprising a piercing member, e.g., a pushable ‘drawing pin’-like member, having a movable, pushable, rotatable or otherwise actuatable head and a nail interconnected with said head and substantially perpendicular to head's plane. When actuated, the nail pierces a separating wall, membrane or the like. The nail shank has at least one crossing aperture, configured to be expose, thus being open, when actuated and piercing said wall or membrane; thus providing a capillary-size open aperture for flowing of homogenized stool fluid from the inner volume of the disposable module to the lower portion of diagnostic strip 314. It is acknowledged that thin-layer chromatography (TLC), filter paper strips and other diagnostic members are utilizable here, see Sherwal, B. L., and Subhash Dadeya. “Filter paper strip kit: a cost-effective transport media.” Tropical doctor 35.2 (2005) and references thereof incorporated herein as a reference.

Reference is now made to FIG. 25, schematically illustrating a perspective view of the disposable module B. Cord (301) is freely yet sealingly reciprocated linearly along cord's main axis, via cork's aperture. The cork (302) is a stopper provided as a top wall of container 4. Rotatable shaft 5 has a main longitudinal axis; one side along said axis is provided within the disposable module where is connected to the proximal end of the cord. Its opposite end (6), located outside disposable module B, is a push-in fitting or push-in connector, configured for fast, reversible and easy connection of the disposable module B, via shaft's push-in connector with the multiuse module A, via motor's axle or gear thereof.

Now for the description with the multiuse module, A. Reference is now made to FIGS. 23, 26, 28 29a & 29b. Multiuse module A comprises, inter alia, at least one motor (308), e.g., electrical motor, and power source, e.g., one or more 1.5V batteries (309), a processor (306) is connected to the motor. On/Off switch (312) is connected to the same. In one example, the motor, batteries and processor are hermetically enveloped by O-ring (311) containing lid (313) of the water-proof housing (307).

According to one embodiments of the invention, multiuse module A further comprises image acquiring means, such as color CCD, or data acquiring sensors, and a processor and communicator configured to acquire the diagnostic results and real-time transmit it to a remote location, to a remote smartphone e.g., via Wi-Fi of Bluetooth™ communication protocols.

Although the detailed descriptions above contain many specifics, these should not be construed as limiting the scope of the invention but as merely providing illustrations of some of the presently preferred embodiments of this invention. Various other embodiments and ramifications are possible within its scope, a number of which are discussed in general terms above. While the invention has been described with a certain degree of particularity, it should be recognized that elements thereof may be altered by persons skilled in the art without departing from the spirit and scope of the invention. Accordingly, the present invention is not intended to be limited to the specific forms set forth herein, but on the contrary, it is intended to cover such alternatives, modifications and equivalents as can be reasonably included within the scope of the invention. The invention is limited only by the following claims and their equivalents.

Although the detailed descriptions above contain many specifics, these should not be construed as limiting the scope of the invention but as merely providing illustrations of some of the presently preferred embodiments of this invention. Various other embodiments and ramifications are possible within its scope, a number of which are discussed in general terms above. While the invention has been described with a certain degree of particularity, it should be recognized that elements thereof may be altered by persons skilled in the art without departing from the spirit and scope of the invention. Accordingly, the present invention is not intended to be limited to the specific forms set forth herein, but on the contrary, it is intended to cover such alternatives, modifications and equivalents as can be reasonably included within the scope of the invention. The invention is limited only by the following claims and their equivalents.

1. A device according to claim 82, comprising
a motor actuating a rotatable axle reversibly connectable to said rotatable shaft, wherein said motor is an integral components within said housing or is a separable module connectable to said housing.

2-81. (canceled)

82. A stool sample collecting device, comprising:
a. a housing including a chamber configured to be reversibly attachable to a toilet-bowl;
b. a retractable swab wounded around a rotatable shaft, wherein said retractable swab is configured to be reversibly installed within said toilet having proximal and distal opposite ends; characterized by at least 2 configurations; an EXTENDED configuration, in which said distal end is configured to be in contact with said stool specimen; and, a RETRACTED configuration, in which said swab's distal end is retracted by rotation of said rotatable shaft into said chamber configuration.

83. A device according to claim 82, comprising at least one detector within said housing and in a fluid connection with said chamber, wherein said at least one detector detects at least one marker in said stool specimen.

84. A device according to claim 83, wherein said at least one detector detects calprotectin in said stool specimen.

85. A device according to claim 83, wherein said at least one detector detects a marker selected from a group consisting of Lactofferin, S100A12, Pancreatic Elastase Anti-Gliadin-IgA-Antibodies, M2PK, sDNA, C. difficile bacteria, Helicobacter pylori, parasites, Candida, leucocytes, Rota virus, Chlamydia, Salmonella, PH and any combination thereof, in said stool specimen.

86. A device according to claim 83, wherein said at least one detector comprises at least one lateral flow strip.

87. A device according to claim 83, wherein said at least one detector comprises one or more color coded diagnosing strips.

88. A device according to claim 82, wherein a portion of said housing is transparent and/or comprises a window.

89. A device according to claim 1, comprising at least one battery, and wherein said motor comprises an electric motor.

90. A device according to claim 89, comprising an optic sensor configured to acquire an image of said at least one detector or an image of fluid in said chamber.

91. A device according to claim 90, comprising a communicator for transmitting said acquired image to a remote location.

92. A device according to claim 82, wherein said retractable swab comprising at least one material selected from a group comprising an abrasive material, an absorbing material, porosive material, layered and multi-laminar material, emulsified material, liposomal material, salt-containing material, buffer-containing material, aggregated material, solid particles, fibers-containing material, filament(s)-containing material, pre-moistened material and any combination thereof.

93. A device according to claim 82, wherein said retractable swab is in the shape of a string, a threaded rope, natural fiber rope, synthetic fiber rope, tube-like, sponge-like thread, projected bulges, a vertebrate-like structure, cord-like thick and/or hairy fiber, dental-floss-like filament having a thin and narrow cross section; filament having grooves, measuring tape-like swab having relatively wide and thin cross section and any combination thereof.

94. A device according to claim 1, wherein said motor comprises a spring.

95. A device according to claim 82, comprising a stool collector along which said retractable swab retracts, wherein said stool collector is configured to be reversibly installed within a toilet-bowl.

96. A device according to claim 95, comprising at least one fastener for fastening said stool collector to said toilet's bowl and/or to said toilet seat.

97. A method of stool sampling, comprising:
installing a portion of a retractable swab within a toilet bowl;
retracting said portion of said retractable swab through stool within said toilet bowl into a chamber of a stool sampling device connected to a proximal end of said retractable swab;
collecting a stool sample by said retracted portion of said retractable swab during said retracting into said chamber, while said stool sampling device is reversibly attached to said toilet bowl.

98. A method according to claim 97, wherein said installing comprises reversibly installing a stool collector along which said retractable swab retracts within said toilet bowl, wherein said stool collector is shaped as a nest having an upper opening.

99. A method according to claim 97, comprising:
detecting at least one marker in a mixture between said buffer and said stool sample using at least one detector in said mobile stool sampling device.

100. A method according to claim 97, wherein said detecting comprises detecting one or more of Calprotectin, Lactofferin, S100A12, Pancreatic Elastase, as well as premier inflammatory and metabolic biomarkers such as EPX and Beta-glucaronidase, Anti-Gliadin-IgA-Antibodies, M2PK, sDNA, C. difficile bacteria, Helicobacter pylori, parasites, Candida, leucocytes, Rota virus, Chlamydia, Salmonella, PH.
